(19) United States
(12) Patent Application Publication
(10) Pub. No.: US 2025/0312289 A1
(43) Pub. Date: Oct. 9, 2025

(54) METHODS AND COMPOSITIONS COMPRISING TOBACCO MILD GREEN MOSAIC VIRUS (TMGMV)

(71) Applicant: The Regents of the University of California, Oakland, CA (US)

(72) Inventors: Nicole Steinmetz, La Jolla, CA (US); Ivonne Gonzalez Gamboa, La Jolla, CA (US)

(21) Appl. No.: 18/872,900

(22) PCT Filed: Jun. 16, 2023
(86) PCT No.: PCT/US2023/025573

§ 371 Date: Dec. 9, 2024

Related U.S. Application Data
(60) Provisional application No. 63/353,309, filed on Jun. 17, 2022.

(51) Int. Cl. A61K 9/51; A01N 25/28; A61K 47/69; B82Y 5/00; C12N 7/00

(52) U.S. Cl. CPC A61K 9/5184; A01N 25/28; A61K 9/5192; A61K 47/6901; C12N 7/00; B82Y 5/00; C12N 2770/00042

(57) ABSTRACT

This application relates in part to nanoparticles comprising a tobamovirus and nanoparticles comprising a tobamovirus and beta-cyclodextrin (β-CD or BCD). This application also relates in part to nanoparticles comprising tobamovirus and one or more active ingredients (AIs) that are non-covalently conjugated to the tobamovirus. The application also provides methods of making and methods of using such nanoparticles as well as compositions comprising the disclosed nanoparticles.

### CROSS-REFERENCE TO RELATED APPLICATIONS

This application claims the benefit of U.S. Provisional Application No. 63/353,309, filed on Jun. 17, 2022. The entire contents of the foregoing are incorporated herein by reference in their entireties.

### FEDERALLY SPONSORED RESEARCH OR DEVELOPMENT

This invention was made with Government support under 2020-67021-31255 and 2022-67012-36698 awarded by the United States Department of Agriculture and under DMR-2011924 awarded by the National Science Foundation. The Government has certain rights in the invention.

### BACKGROUND

Pesticides are extensively used for food production in field. But pesticides and methods used to apply them are inefficient. Pesticides accumulate in the environment, on crops, and in drinking water. Pesticides are toxic to the environment and human health. It important to develop better ways to apply pesticides. The extensive use of pesticides in agriculture causes these toxins to accumulate on crops, in soil, as well as in drinking water and groundwater, severely endangering the ecosystem and human health. The first step toward a healthier society is to enhance food security by improving quality and yields (i.e., more effective crop treatment), while protecting the environment and agricultural ecosystems (i.e., preventing the leaching and accumulation of pesticides in the environment). Most pesticides are hydrophobic and thus do not have good soil mobility. This leads to overuse and consequently increased health and environmental problems. It important to develop better ways to apply pesticides.

### SUMMARY

This application is based, in part, on the surprising discovery that tobamovirus (e.g., tobacco mild green mosaic virus (TMGMV)) rods can be used to load (also called encapsulate throughout) a target active ingredient (AI, also called active substance), such as pesticides, drugs, and pharmaceuticals. Importantly, the compositions and methods described herein do not require any modification to any useful drug, pesticide, pharmaceutical, or compound. In part, this application involves non-covalent encapsulation or loading techniques to encapsulate pesticides and/or drugs into the nanoparticles described herein. Tobamovirus rods are a good platform for precision farming, because they have excellent soil mobility, and described herein, the nanoparticles created using a tobamovirus can have even soil distribution and/or soil mobility, up to and above 30 cm. In some embodiments, the nanoparticles of the disclosure have a soil distribution and/or soil mobility of at least 5, 10, 15, 20, 21, 22, 23, 24, 25, 26, 27, 28, 29, 30, 21, 32, 33, 34, 35, 36, 37, 38, 39, or 40 cm.

Described herein, inter alia, is the use of non-covalent encapsulation techniques to encapsulate pesticides onto the nanoparticles comprising tobamovirus (tobacco mild green mosaic virus (TMGMV)). Tobamovirus nanoparticles are a good platform for precision farming because they have excellent soil mobility. The present application includes tobamovirus nanoparticles that use beta-cyclodextrin (also called BCD, bCD, βCD, β-CD, etc. throughout the disclosure) as a cargo pocket, and tobamovirus nanoparticles capable of undergoing structural transitions that enable molecule (e.g., AI) infusion into the tobamovirus structure. In some embodiments, the BCD is conjugated to the surface tobamovirus nanoparticles; without being bound by theory, the BCD functions as a pocket to load cargo, such as medical drugs and pesticides. In some embodiments, the structural transitions that enable molecule (e.g., AI) infusion in the tobamovirus nanoparticles are triggered by an external factor. In some embodiments, the external factor is exposing the tobamovirus nanoparticles to a pH change or a solvent (e.g., dimethylsulfoxide or DMSO).

While often depicted as rigid/solid structures, plant viruses including tobamoviruses “breathe” in solution and through careful adjustment of pH, the structures can be opened to encapsulate at least one active ingredients or one or more active ingredients. Described herein, inter alia, are methods for triggering a tobamovirus nanoparticle to partially and reversibly dissociate one or more of its coat proteins by adjusting the pH or by contacting the tobamovirus nanoparticles with a solvent (e.g., DMSO). Such methods allow “breathing” of tobamovirus or a phase transition that allows the infusion of drug molecules or any AI into the tobamovirus structure. In the present disclosure, the AI can be one or more of pesticides and other drugs.

The disclosed nanoparticles have good soil mobility and, in some embodiments, the nanoparticles utilize pesticide loading two strategies (1) β-CD as a cargo pocket for an AI, and (2) structural transitions for molecule and/or AI infusion. In some embodiments, beta-cyclodextrin (β-CD) is conjugated to the surface of tobamovirus nanoparticles; without being bound by theory, the β-CD functions as a pocket to load cargo/AI, such as medical drugs and pesticides.

Also without being bound by theory, while often depicted as rigid/solid structures, plant viruses including tobamovirus “breathe” in solution and through careful adjustment of pH, and the structure can be opened to entrap one or more AI. As described herein, a breathing method for tobamovirus that allows it to undergo structural transitions and to infuse drug molecules into the structure has been developed. As described herein, the breathing method can be used to entrap or infuse multiple AIs, including pesticides and other drugs, in the tobamovirus nanoparticles. In some embodiments, the compositions and the methods described herein utilize supramolecular interactions between β-cyclodextrin and target AIs to formulate multifunctional nanoparticles for delivery applications.

B-cyclodextrin is a natural toroid-shaped cyclic oligosaccharide. It has a hydrophilic exterior surface and a hydrophobic interior cavity that can accommodate a broad range of guest molecules. Furthermore, it is the most widely used host-system in supramolecular chemistry, as well as low cost, with good water solubility and biocompatible properties. Without being bound by theory, the rationale is to use a supramolecular strategy based on the interaction between β-CD and target A.I.s (e.g., pesticides). B-CD units are grafted onto the exterior surface of tobamovirus using optimized bioconjugation reactions that will capture target one or more AIs for efficient delivery into soil.

In some embodiments, nanoparticles described herein entrap pesticides into tobamovirus by pH change which entraps AIs through the formation of “pockets” or “pores” between CPs.

The rationale is that by increasing the pH of the buffer or by the presence of a solvent (e.g., DMSO), the virus will start to dissociate and hydrophobic pockets or pores will be created between the virion's coat proteins. In an exemplary method, AIs are then added to interact with the virus particles and then the pH is decreased to promote particles' self-assembly and entrapment of AI on the hydrophobic pockets or pores. In yet another exemplary method, AIs are added to interact with the virus particles after the addition of a solvent (e.g., DMSO) to promote entrapment of AI on the hydrophobic pockets or pores.

Certain aspects of the present disclosure are directed to a nanoparticle comprising a tobamovirus; and one or more active ingredients (AIs) that are non-covalently conjugated to the tobamovirus, wherein the tobamovirus comprises one or more coat proteins that reversibly and partially dissociate in response to an external factor.

In some embodiments, the one or more coat proteins reversibly and partially dissociate to form one or more pores. In some embodiments, the one or more AIs are non-covalently conjugated to and entrapped within the one or more pores of the tobamovirus. In some embodiments, the one or more AIs are intercalated in the one or more coat proteins of the tobamovirus. In some embodiments, the one or more AIs are not chemically altered. In some embodiments, the external factor is a change in pH. In some embodiments, the external factor is the presence of a solvent. In some embodiments, the solvent is a polar, aprotic solvent. In some embodiments, the solvent is a polar, aprotic solvent that is miscible with water. In some embodiments, the polar, aprotic solvent is dimethylsulfoxide (DMSO). In some embodiments, the TM tobamovirus GMV is rod-shaped.

In some embodiments, described herein are nanoparticles comprising a tobamovirus and a beta-cyclodextrin (OCD). In some embodiments, the nanoparticle further comprises an R group between the tobamovirus and the βCD. In some embodiments, the tobamovirus and the βCD are covalently linked. In some embodiments, the tobamovirus and the βCD are linked with an R group. In some embodiments, the R group is an alkyl, alkene, alkyne, ester, or other carbon-containing compound. In some embodiments, the R group is ethyne.

In some embodiments, the tobamovirus-AI nanoparticle has a width that is larger than the width of a reference tobamovirus. In some embodiments, the reference tobamovirus molecule is treated with the same conditions as the tobamovirus-AI nanoparticle without the addition of an AI. In some embodiments, this application relates to nanoparticles comprising a tobamovirus and one or more active ingredient (AIs), wherein the width of the tobamovirus-AI nanoparticle is larger than a reference. In some embodiments, the reference is the width of a tobamovirus molecule treated in the same conditions without the addition of an AI. In some embodiments, the reference is 15, 16, 17, or 18 nm. In some embodiments, the width of the tobamovirus-AI nanoparticle is 2%-105% larger than that of the reference. In some embodiments, the width of the tobamovirus-AI nanoparticle is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 9, 95, 96, 97, 98, 99, 100, 101, 102, 103, 104, or 105% larger than that of the reference. In some embodiments, the width of the tobamovirus-AI nanoparticle is 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51. In some embodiments, the one or more AI comprises one or more of a drug, pesticide, or a small molecule. In some embodiments, the pesticide is a water-insoluble organic compound, an insecticide, a herbicide, a fungicide, an acaricide, an algicide, an antimicrobial agent, biopesticide, a biocide, a disinfectant, a fumigant, an insect growth regulator, a plant growth regulator, a miticide, a microbial pesticide, a molluscide, a nematicide, an ovicide, a pheromone, a repellent, a rodenticide, a defoliant, a desiccant, a safener, or any combination thereof. In some embodiments, the pesticide is a benzoyl urea, such as novaluron, lufenuron, chlorfluazuron, flufenoxuron, hexaflumuron, noviflumuron, teflubenzuron, triflumuron and diflubenzuron; a carbamate; a pyrethroid, such as cyhalothrin and isomers and isomer mixtures thereof, lambda-cyhalothrin, deltamethrin, tau-fluvalinate, cyfluthrin, beta-cyfluthrin, tefluthrin, and bifenthrin; an organophosphate, such as azinfos-methyl, chlorpyrifos, diazinon, endosulfan, methidathion; a neonicotinoid; a phenylpyrazole, such as imidacloprid, acetamiprid, thiacloprid, dinotefuran, thiamethoxam, and fipronil; a conazole, such as epoxiconazole, hexaconazole, propiconazole, prochloraz, imazalil, triadimenol, difenoconazole, myclobutanil, prothioconazole, triticonazole, and tebuconazole; a morpholine, such as dimethomorph, fenpropidine, and fenpropimorph; a strobilurin, such as azoxystrobin, kresoxim-methyl, and analogues thereof; a phthalonitrile, such as chlorothalonil; a mancozeb; a fluazinam; a pyrimidine, such as bupirimate; an aryloxyphenoxy derivative; an aryl urea; an aryl carboxylic acid; an aryloxy alkanoic acid derivative, such as clodinafop-propargyl and analogues thereof, fenoxaprop-p-ethyl and analogues thereof, propaquizafop, quizalafop and analogues thereof; a dintroaniline, such as pendimethalin and trifluralin; a diphenyl ether, such as oxyfluorfen; an imidazolinone; a sulfonylurea, such as chlorsulfuron, nicosulfuron, rimsulfuron, tribenuron-methyl; a sulfonamide; a triazine; and a triazinone, such as metamitron.

In some embodiments, at least one AI comprises at least one of a drug, pesticide, or a small molecule. In some embodiments, the drug can be a chemokine, an antibacterial, or any therapeutic compound. In some embodiments, the drug is a chemotherapeutic drug, an antiparasitic drug, an antibiotic drug, or an immunomodulator. In some embodiments, the drug is a hydrophilic drug or a hydrophobic drug. In some embodiments, the tobamovirus is a Tobacco Mild Green Mosaic Virus (TMGMV). In some embodiments, the tobamovirus is a Tobacco Mosaic Virus (TMV).

In some embodiments, the nanoparticle comprises about 1 to about 1500 AI molecules per tobamovirus.

Also described herein are compositions comprising any disclosed nanoparticle. In some embodiments, any nanoparticle or composition described herein has a soil distribution and/or soil mobility is at least 5, 10, 15, 20, 21, 22, 23, 24, 25, 26, 27, 28, 29, 30, 21, 32, 33, 34, 35, 36, 37, 38, 39, or 40 cm. In some embodiments, the composition further comprises an excipient. In some embodiments, the excipient is a buffer or water.

Disclosed herein, in certain embodiments, are methods of making a nanoparticle comprising a tobamovirus and a βCD, the method comprising: providing isolated tobamovirus, coupling the βCD to the tobamovirus, thereby creating the nanoparticle; and purifying the nanoparticle.

In some embodiments, the coupling step comprises forming a covalent bond between the βCD, a linker, and the tobamovirus. In some embodiments, the coupling step comprises a diazonium coupling reaction. In some embodiments of any of the compositions or methods described herein, the tobamovirus is modified or inactivated. In some embodiments, the linker is an R group. In some embodiments, the R group is an alkyl, alkene, alkyne, ester, or other carbon-containing compound. In some embodiments, the R group is ethyne.

Also disclosed herein, in certain embodiments, are methods of making a nanoparticle comprising tobamovirus and one or more active ingredients (AIs), the method comprising: providing isolated tobamovirus a buffer having a pH of about 7 to 9; adding one or more AIs to the tobamovirus more than once, thereby creating the nanoparticle; purifying the nanoparticle in a solution having a pH of about 5 to 9, wherein, the one or more AIs are non-covalently conjugated to the tobamovirus, and wherein the tobamovirus comprises one or more coat proteins that reversibly and partially dissociate in response to a change in pH.

In some embodiments, the one or more AIs are added for at least 1, 2, 3, 4, 5, 6, 7, 8, 9, 10, 11, 12, 13, 14, 15, 16, 17, 18, 19, 20, or 21 days. In some embodiments, the buffer has a pH of about 7 to 7.5, 7.5 to 8, 7 to 8, 8 to 8.5, 8.5 to 9, or 8 to 9. In some embodiments, the pH of the buffer is 7.2-7.8, 7.3-7.8, 7.2-7.7, 7.3-7.7, 7.4-7.8, 7.4-7.7, 7.5-7.7, 7.5-7.8, 7.2-7.6, 7.3-7.6, 7.4-7.6, 7.5-7.6, 7.2-7.5, 7.3-7.5, 7.4-7.5, 7.2-7.9, 7.3-7.9, 7.4-7.9, 7.5-7.9, 7.3-7.99, 7.4-7.99, or 7.5-7.99; or wherein the buffer has a pH of about 7.2, 7.3, 7.4, 7.5, 7.6, 7.7, 7.8, 7.9, or 7.99. In some embodiments, the solution has a pH of about 6.9, 7.0, 7.1, 7.2, or 7.3. 1. In some embodiments, the change in pH is about 0.5 to 1, about 0.5 to 2, 0.5 to 3, 1 to 2, or 1 to 3.

Disclosed herein, in certain embodiments, are method of making a nanoparticle comprising tobamovirus and one or more active ingredients (AIs), the method comprising: providing isolated tobamovirus to a buffer having a pH of about 5 to 9 to create a tobamovirus-buffer; adding a solvent at a concentration of about 15% (v/v) to about 25% (v/v); adding one or more AIs to the tobamovirus-buffer, thereby creating the nanoparticle; and purifying the nanoparticle in a solution having a pH of about 5 to 9, wherein, the one or more AIs are non-covalently conjugated to the tobamovirus, and wherein the tobamovirus comprises one or more coat proteins that reversibly and partially dissociate in response to the presence of the solvent.

In some embodiments, the solvent is added dropwise. In some embodiments, the one or more AIs are added dropwise. In some embodiments, the one or more AIs are added stepwise over a period of time. In some embodiments, the period of time is about 0.5 hours to about 10 days. In some embodiments, the one or more AIs are added once a day. In some embodiments, the methods further comprise incubating the one or more AIs in the tobamovirus-buffer for about 4 hours to about 24 hours. In some embodiments, the solvent is a polar, aprotic solvent. In some embodiments, the polar, aprotic solvent is dimethylsulfoxide (DMSO). In some embodiments, the one or more coat proteins reversibly and partially dissociate to form one or more pores.

In some embodiments, the one or more AIs are added repetitively. In some embodiments, the one or more AIs are added to the tobamovirus-buffer two or more times. In some embodiments, the one or more AIs are added at least once a day. In some embodiments, the one or more AIs are added until reaching an equivalence ratio of about 10:1, 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, or 1000:1; or wherein the one or more AIs is added in 1,000, 1,500, 2,000, 2,500, 3,000, 3,3,00, 4,000, 4,500, 5,000, 5,500, 6,500, 7,000, 7,500, 8,000, 8,500, 9,000, or 9,500-fold molar excess to the tobamovirus; or wherein 100, 150, 200, 250, 300, 350, 400, 450, or 500 nmol of one or more Als per gram of tobamovirus is added.

In some embodiments, the one or more AIs are non-covalently conjugated to and entrapped within the one or more pores of the tobamovirus. In some embodiments, the one or more AIs are intercalated in the one or more coat proteins of the tobamovirus. In some embodiments, the one or more AIs are not chemically altered. In some embodiments, the tobamovirus is rod-shaped.

In some embodiments, the nanoparticle has a width larger than the width of a reference tobamovirus. In some embodiments, the reference tobamovirus molecule is treated in the same conditions as the tobamovirus-AI nanoparticle without the addition of an AI. In some embodiments, the reference is 15, 16, 17, or 18 nm. In some embodiments, the width of the nanoparticle is 2%-105% larger than that of the reference; or wherein the width of the nanoparticle is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 9, 95, 96, 97, 98, 99, 100, 101, 102, 103, 104, or 105% larger than that of the reference. In some embodiments, the width of the nanoparticle is 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nm.

In some embodiments, the one or more AI comprises one or more of a drug, pesticide, or a small molecule. In some embodiments, the pesticide is a water-insoluble organic compound, an insecticide, a herbicide, a fungicide, an acaricide, an algicide, an antimicrobial agent, biopesticide, a biocide, a disinfectant, a fumigant, an insect growth regulator, a plant growth regulator, a miticide, a microbial pesticide, a molluscide, a nematicide, an ovicide, a pheromone, a repellent, a rodenticide, a defoliant, a desiccant, a safener, or any combination thereof. In some embodiments, the pesticide is a benzoyl urea, such as novaluron, lufenuron, chlorfluazuron, flufenoxuron, hexaflumuron, noviflumuron, teflubenzuron, triflumuron and diflubenzuron; a carbamate; a pyrethroid, such as cyhalothrin and isomers and isomer mixtures thereof, lambda-cyhalothrin, deltamethrin, tau-fluvalinate, cyfluthrin, beta-cyfluthrin, tefluthrin, and bifenthrin; an organophosphate, such as azinfos-methyl, chlorpyrifos, diazinon, endosulfan, methidathion; a neonicotinoid; a phenylpyrazole, such as imidacloprid, acetamiprid, thiacloprid, dinotefuran, thiamethoxam, and fipronil; a conazole, such as epoxiconazole, hexaconazole, propiconazole, prochloraz, imazalil, triadimenol, difenoconazole, myclobutanil, prothioconazole, triticonazole, and tebuconazole; a morpholine, such as dimethomorph, fenpropidine, and fenpropimorph; a strobilurin, such as azoxystrobin, kresoxim-methyl, and analogues thereof; a phthalonitrile, such as chlorothalonil; a mancozeb; a fluazinam; a pyrimidine, such as bupirimate; an aryloxyphenoxy derivative; an aryl urea; an aryl carboxylic acid; an aryloxy alkanoic acid derivative, such as clodinafop-propargyl and analogues thereof, fenoxaprop-p-ethyl and analogues thereof, propaquizafop, quizalafop and analogues thereof; a dintroaniline, such as pendimethalin and trifluralin; a diphenyl ether, such as oxyfluorfen; an imidazolinone; a sulfonylurea, such as chlorsulfuron, nicosulfuron, rimsulfuron, tribenuron-methyl; a sulfonamide; a triazine; and a triazinone, such as metamitron.

In some embodiments, the drug is a chemotherapeutic drug, an antiparasitic drug, an antibiotic drug, or an immunomodulator. In some embodiments, the drug is a hydrophilic drug or a hydrophobic drug. In some embodiments, the nanoparticle comprises about 1 to about 1500 AI molecules per tobamovirus. In some embodiments, the tobamovirus is a Tobacco Mild Green Mosaic Virus (TMGMV). In some embodiments, the tobamovirus is a Tobacco Mosaic Virus (TMV).

Also described herein are methods comprising administering any nanoparticle or composition described herein to soil, crops, or plants, wherein the nanoparticle or composition is administered in an effective amount.

Also described herein are pharmaceutical compositions comprising any of the nanoparticle of the disclosure.

In some embodiments, the pharmaceutical compositions further comprise at least one pharmaceutically acceptable carrier, diluent, or excipient. In some embodiments, the pharmaceutical composition is formulated into a dosage form that is an injectable solution, a lyophilized powder, a suspension, or any combination thereof.

Also described herein are methods of treating cancer in a subject in need thereof, the method comprising administering a nanoparticle of the disclosure or a pharmaceutical composition of the disclosure to the subject in need of treatment for cancer, wherein the nanoparticle or the composition is administered in an effective amount.

In some embodiments, the cancer wherein the cancer comprises breast cancer, ovarian cancer, glioma, gastrointestinal cancer, prostate cancer, carcinoma, lung carcinoma, hepatocellular carcinoma, testicular cancer, cervical cancer, endometrial cancer, bladder cancer, head and neck cancer, lung cancer, gastro-esophageal cancer, gynecological cancer, or any combination thereof.

Also described herein are methods of treating an infection in a subject in need therefor, the method comprising: administering a nanoparticle of the disclosure or a pharmaceutical composition of the disclosure to the subject in need of treatment for the infection, wherein the nanoparticle or the composition is administered in an effective amount.

In some embodiments, the infection is a bacterial infection, a viral infection, a fungal infection, a parasitic infection, or any combination thereof.

Further on, the disclosure also relates to methods of combating harmful insects and/or phytopathogenic fungi, which comprises contacting plants, soil or habitat of plants in or on which the harmful insects and/or phytopathogenic fungi are growing or may grow, plants or soil to be protected from attack or infestation by said harmful insects and/or phytopathogenic fungi with an effective amount of the formulation according to the present disclosure. The formulations according to the present disclosure can therefore be used for the control of a multitude of phytopathogenic fungi or insects on various cultivated plants or weeds in, such as wheat, rye, barley, oats, rice, com, grass, bananas, cotton, soy, coffee, sugar cane, vines, fruits and omamental plants, and vegetables, such as cucumbers, beans, tomatoes, potatoes and cucurbits.

The present disclosure also relates to methods of controlling undesired vegetation, which comprises allowing a herbicidal effective amount of the formulation according to the present disclosure to act on plants, their habitat. The control of undesired vegetation is understood as meaning the destruction of weeds. Weeds, in the broadest sense, are understood as meaning all those plants which grow in locations where they are undesired.

The formulations and compositions according to the present disclosure can therefore be used for the control of a multitude of phytopathogenic fungi or insects on various cultivated plants or weeds in, such as wheat, rye, barley, oats, rice, corn, grass, bananas, cotton, soy, coffee, sugar cane, vines, fruits and ornamental plants, and vegetables, such as cucumbers, beans, tomatoes, potatoes and cucurbits, and on the seeds of these plants.

Thus, the formulations according to the present disclosure and compositions according to the present disclosure are suitable for controlling common harmful plants in useful plants, in particular in crops such as oat, barley, millet, corn, rice, wheat, sugar cane, cotton, oilseed rape, flax, lentil, sugar beet, tobacco, sunflowers and soybeans or in perennial crops.

Embodiments disclosed below include nanoparticles including a tobamovirus and one or more active ingredients (AIs) that are non-covalently conjugated to the tobamovirus, compositions containing these nanoparticles, methods of using these nanoparticles, and methods of preparing these therapeutic nanoparticles. Some embodiments of the nanoparticles, compositions, and methods described herein may provide one or more of the following advantages.

First, certain embodiments of the present disclosure include nanoparticles and compositions that can be prepared efficiently and in a cost-effective manner. Covalent conjugation is often used in the preparation of pesticides and nanoparticles. However, covalent conjugation is a complex and resource-intensive process, and pesticides are challenging to conjugate to proteins given their high degree of hydrophobicity. Furthermore, the costs and regulatory processes associated with using covalent conjugation strategies for pesticide linking to nanoparticles (including classifying, characterizing, and approving covalently modified chemicals) may outweigh the benefits for agricultural use. Thus, an efficient non-covalent method of loading nanoparticles is desired. The nanoparticles and compositions of the disclosure address this need by including AIs that do not need to be chemically altered and that can be efficiently loaded or infused into the tobamovirus nanoparticles without the need for covalent conjugation.

Second, certain embodiments of the present disclosure include nanoparticles and compositions that can be used for a wide variety of applications depending on which AI is selected and loaded into the tobamovirus nanoparticles. For example, in some embodiments, the nanoparticles and compositions of the disclosure can be used to treat a disease in a subject in need thereof, to combat harmful insects and/or phytopathogenic fungi, and to control undesired vegetation.

Third, certain embodiments of the present disclosure include nanoparticles that can have a high AI loading efficiency. For example, in some embodiments, the nanoparticles and compositions of the disclosure can be loaded with about 1100 molecules of AI or more per virion.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic depiction of the formation of the TMGMV-βCD (top row) and the loading of an AI onto the TMGMV-βCD (bottom row).

FIG. 2 is an SDS-PAGE gel that showed the size of the TMGMV (lane 1), TMGMV-alkyne (lane 2), and the TMGMV-βCD (lane 3).

FIG. 3 is graph of size exclusion chromatography (SEC) that showed the elution of TMGMV and TMGMV-βCD.

FIG. 4 is a transmission electron microscopy (TEM) that showed the β-CD-TMGMV after purification. The bar represents a size of 200 nm.

FIG. 5 is a schematic depiction of the method used to detect and quantify loading of AI onto β-CD-TMGMV.

FIG. 6 is a table of the results from the doxorubicin (“DOX”) displacement from β-CD-TMGMV by the addition of Clothianidin (“CTD”), Fluopyram (“FLP”), or Tetracycline (“TET”).

FIG. 7 is a schematic depiction of the “breathing” phase transition diagram, which illustrates the effect of pH on active ingredient (AI) entrapment into TMGMV.

FIG. 8 is an Image J analysis of a TEM image depicting measurements taken of a TMGMV construct. Images of TMGMV (control) and TMGMV infused with doxorubicin, ATTO550, fluopyram and clothianidin were taken and analyzed.

FIGS. 9A-9F revealed the increase in nanoparticle width the TMGMV constructs. TEM images showed the TMGMV infused with doxorubicin (FIG. 9A; bar represents a size of 50 nm), fluopyram (FIG. 9B; bar represents a size of 100 nm), clothianidin (FIG. 9C; bar represents a size of 50 nm), ATTO550 (FIG. 9D; bar represents a size of 100 nm), and TMGMV alone (FIG. 9E; control; bar represents a size of 500 nm) as well as a bar graph showing the average width of each of the TMGMV constructs (FIG. 9F).

FIGS. 10A-10B are schematic depictions of the experimental set up for the soil column (FIG. 10A) and soil mobility analysis (FIG. 10B).

FIGS. 11A-11C show the distribution of TMGMV and infused TMGMV.

FIG. 11A are SDS gels that showed which soil fractions that contained the TMGMV (top gels) and the infused TMGMV (bottom gels). The gels were quantified and the soil fractions that contained the TMGMV (FIG. 11B) and the infused TMGMV (FIG. 11C) were plotted.

FIGS. 12A-12B are schematic depictions of the assays used for soil mobility analysis using SDS-PAGE (FIG. 12A) and a plate reader (FIG. 12B).

FIGS. 13A-13B show the soil mobility of TMGMV infused with doxorubicin. FIG. 13A is a graph of the portion of dye in the soil fractions presented as a percentage of the total dye in the soil. FIG. 13B are the results of the SDS-PAGE (top gels) and the plate reader (bottom gels) that showed the presence of the infused TMGMV in the soil fractions.

FIGS. 14A-14B show the soil mobility of TMGMV infused with Cy5 amine. FIG. 14A is a graph of the portion of dye in the soil fractions presented as a percentage of the total dye in the soil. FIG. 14B are the results of the SDS-PAGE (top gels) and the plate reader (bottom gels) that showed the presence of the infused TMGMV in the soil fractions.

FIG. 15 is a graph of absorbance at 646 nm in soil fractions treated with Cy5 Amine alone or with TMGMV-βCD loaded with Cy5 amine.

FIGS. 16A-16C are schematic depictions of the methodology for infusion of the active ingredient (AI) into the TMGMV nanoparticles. FIG. 16A is a schematic depiction of the steps to infuse the AI into the TMGMV nanoparticles via the pH approach. FIG. 16B is a schematic depiction of the steps to infuse the AI into the TMGMV nanoparticles via the dimethylsulfoxide (DMSO) approach. FIG. 16C is a schematic depiction of the characterization steps of the AI-loaded TMGMV nanoparticles.

FIGS. 17A-17B show images and imaging quantification of TMGMV nanoparticles infused (i.e., non-covalently conjugated) with various active ingredients (AI). FIG. 17A are TEM images of the TMGMV nanoparticles loaded with fluopyram, clothianidin, ivermectin, and rifampicin. FIG. 17B are graphs showing Image J analysis of the TEM images of FIG. 17A depicting measurements taken of the AI-infused TMGMV nanoparticles; ****p-value is <0.00001.

FIGS. 18A-18B are graphs showing circular dichroism spectra for non-covalently loaded TMGMV samples. FIG. 18A is a graph showing circular dichroism spectra for TMGMV nanoparticles infused with AI via the pH method. FIG. 18B is a graph showing circular dichroism spectra for TMGMV nanoparticles infused with AI via the DMSO method.

FIGS. 19A-19J are graphs showing image analysis from transmission electron microscopy comparing the length of virions after AI infusion. FIG. 19A is a graph summarizing the quantification of virions and their lengths of AI-infused TMGMV nanoparticles prepared via the pH method. FIGS. 19B-19E are graphs showing the quantification of virions and their lengths of TMGMV nanoparticles infused with clothianidin (FIG. 19B), ivermectin (FIG. 19C), fluopyram (FIG. 19D), and rifampicin (FIG. 19E) via the pH method. FIG. 19F is a graph summarizing the quantification of virions and their lengths of AI-infused TMGMV nanoparticles prepared via the DMSO method. FIGS. 19G-19J are graphs showing the quantification of virions and their lengths of TMGMV nanoparticles infused with clothianidin (FIG. 19G), ivermectin (FIG. 19H), fluopyram (FIG. 19I), and rifampicin (FIG. 19J) via the DMSO method.

FIGS. 20A-20D are illustrations showing the surface charge distribution of various AI molecules. FIG. 20A shows the surface charge distribution of clothianidin. FIG. 20B shows the surface charge distribution of fluopyram. FIG. 20C shows the surface charge distribution of ivermectin. FIG. 20D shows the surface charge distribution of rifampicin.

FIGS. 21A and 21B are images of molecular modeling simulations of the molecular docking of TMGMV coat proteins and rifampicin.

FIGS. 22A and 22B are images of molecular modeling simulations of the molecular docking of TMGMV coat proteins and ivermectin.

FIGS. 23A and 23B are images of molecular modeling simulations of the molecular docking of TMGMV coat proteins and fluopyram.

FIGS. 24A and 24B are images of molecular modeling simulations of the molecular docking of TMGMV coat proteins and clothianidin.

FIG. 25 is a TEM image of TMGMV particle aggregation after AI infusion (e.g., Cy5).

FIGS. 26A-26D show the characterization of AI-infused TMGMV nanoparticles additionally loaded with cyanine5 (Cy5) and doxorubicin. FIG. 26A is a transmission electron microscopy (TEM) image of AI-infused TMGMV nanoparticles loaded with Cy5. FIG. 26B is a graph showing size exclusion chromatography of AI-infused TMGMV nanoparticles loaded with Cy5. FIG. 26C is a TEM image of AI-infused TMGMV nanoparticles loaded with doxorubicin. FIG. 26D is a graph showing size exclusion chromatography of AI-infused TMGMV nanoparticles loaded with doxorubicin.

FIG. 27 is a graph showing the width comparison of TMGMV nanoparticles prepared via the pH and DMSO methods. Wild type TMGMV was exposed to pH 7.5 and 20% DMSO concentration, as described in the “Methods” section of the Examples and in Example 6. Transmission electron microscopy was performed and TMGMV nanoparticles were analyzed with ImageJ software to determine the particles' length (n=150).

FIGS. 28A-28D are graphs showing the heats of binding for each conformation of docked AIs on TMGMV and their implicated residues, as calculated using a molecular modeling simulation software. FIG. 28A is a graph showing the heats of binding for each conformation of docked ivermectin on TMGMV and its implicated residues. FIG. 28B is a graph showing the heats of binding for each conformation of docked fluopyram on TMGMV its implicated residues. FIG. 28C is a graph showing the heats of binding for each conformation of docked clothianidin on TMGMV and its implicated residues. FIG. 28D is a graph showing the heats of binding for each conformation of docked rifampicin on TMGMV and its implicated residues.

FIGS. 29A-29D are tables showing the regions of binding of clothianidin (FIG. 29A), fluopyram (FIG. 29B), ivermectin (FIG. 29C), and rifampicin (FIG. 29D), their function for TMGMV, and the residues specifically identified to stabilize these AIs.

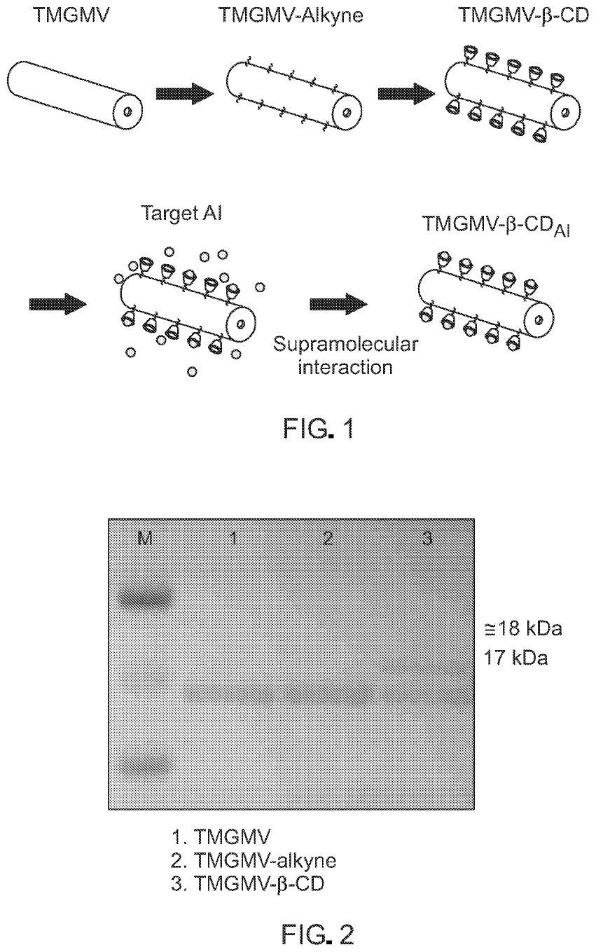

### DETAILED DESCRIPTION

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the” include plural referents unless the content clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±15%, ±10%, ±5%, ±4%, ±3%, ±2%, or ±1% are within the intended meaning of the recited value.

By the term “nanoparticle” is meant an object that has a length between about 2 nm to about 300 nm (e.g., between about 2 nm and 100 nm, between 2 nm and 200 nm, between 2 nm and 250 nm, between 2 nm and 300 nm, between 100 nm and 200 nm, between 100 nm and 250 nm, between 100 nm and 300 nm, between 150 nm and 250 nm, between 200 nm and 300 nm, between 200 nm and 250 nm). Non-limiting examples of nanoparticles include the nanoparticles described herein.

The terms “subject” or “patient,” as used herein, refer to any mammal (e.g., a human or a veterinary subject, e.g., a dog, cat, horse, cow, goat, sheep, mouse, rat, or rabbit) to which a composition or method of the present disclosure may be administered to, e.g., for experimental, diagnostic, prophylactic, and/or therapeutic purposes. The subject may seek or need treatment, require treatment, is receiving treatment, will receive treatment, or is under care by a trained professional for a particular disease or condition.

By the term “chemotherapeutic agent” is meant a molecule that can be used to reduce the rate of cancer cell growth or to induce or mediate the death (e.g., necrosis or apoptosis) of cancer cells in a subject (e.g., a human). In non-limiting examples, a chemotherapeutic agent can be a small molecule, a protein (e.g., an antibody, an antigen-binding fragment of an antibody, or a derivative or conjugate thereof), a nucleic acid, or any combination thereof. Non-limiting examples of chemotherapeutic agents include: cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, azacitidine, axathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

As described herein, the term “effective amount” is an amount which brings about a desired result. A desired result in the context of this description can comprise a reduction in an undesired characteristic (e.g., a reduction in the undesired organism, reduction in undesired plant, etc.).

This document provides nanoparticles, compositions, methods of making, and methods of applying engineered tobamoviruses for the purpose of drug and pesticide delivery. The nanoparticles, compositions, methods of making, and methods of use described herein can include any species of tobamoviruses. For example, the nanoparticles, compositions, methods of making, and methods of use described herein can include tobacco mild green mosaic viruses (TMGMV). In another example, nanoparticles, compositions, methods of making, and methods of use described herein can include the tobacco mosaic viruses (TMV). In some embodiments, the nanoparticles, compositions, methods of making, and methods of use described herein can include a bell pepper mottle virus (BPeMV), brugmansia mild mottle virus, cactus mild mottle virus (CMMoV), clitoria yellow mottle virus, cucumber fruit mottle mosaic virus, cucumber green mottle mosaic virus (CGMMV), cucumber mottle virus, frangipani mosaic virus (FrMV), hibiscus latent Fort Pierce virus (HLFPV), hibiscus latent Singapore virus (HLSV), kyuri green mottle mosaic virus, maracuja mosaic virus (MarMV), obuda pepper virus (ObPV), odontoglossum ringspot virus (ORSV), opuntia chlorotic ringspot virus, paprika mild mottle virus, passion fruit mosaic virus, pepper mild mottle virus (PMMoV), plumeria mosaic virus, rattail cactus necrosis-associated virus (RCNaV), rehmannia mosaic virus, ribgrass mosaic virus (HRV), sammons's Opuntia virus (SOV), streptocarpus flower break virus, sunn-hemp mosaic virus (SHMV), tobacco latent virus, tomato brown rugose fruit virus (ToBRFV), tomato mosaic virus (ToMV), tomato mottle mosaic virus, tropical soda apple mosaic virus, turnip vein-clearing virus (TVCV), ullucus mild mottle virus, wasabi mottle virus (WMoV), yellow tailflower mild mottle virus, youcai mosaic virus (YoMV) aka oilseed rape mosaic virus (ORMV), zucchini green mottle mosaic virus, or any combination thereof.

Tobamovirus is a genus of positive-strand RNA viruses in the family Virgaviridae. TMGMV and TMV are a members of the tobamoviruses, which consist of rod-shaped, RNA viruses that are strictly plant pathogens.

TMGMV and TMV each have 2,130 identical coat proteins arranged helically around a single-stranded RNA genome to form a hollow rigid rod that measures 300×18 nm with a 4 nm internal channel. The external surface of a coat protein features two solvent-exposed tyrosine side chains (Tyr 2 and Tyr 139), which can be functionalized using diazonium coupling reactions. In some embodiments, the tobamovirus (e.g., TMGMV or TMV) is coupled to a carrier, such as beta-cyclodextrin (BCD).

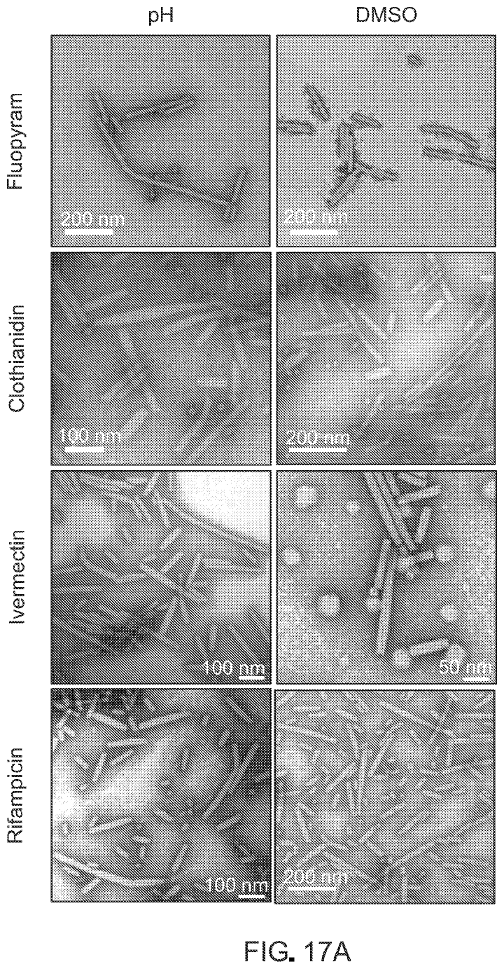

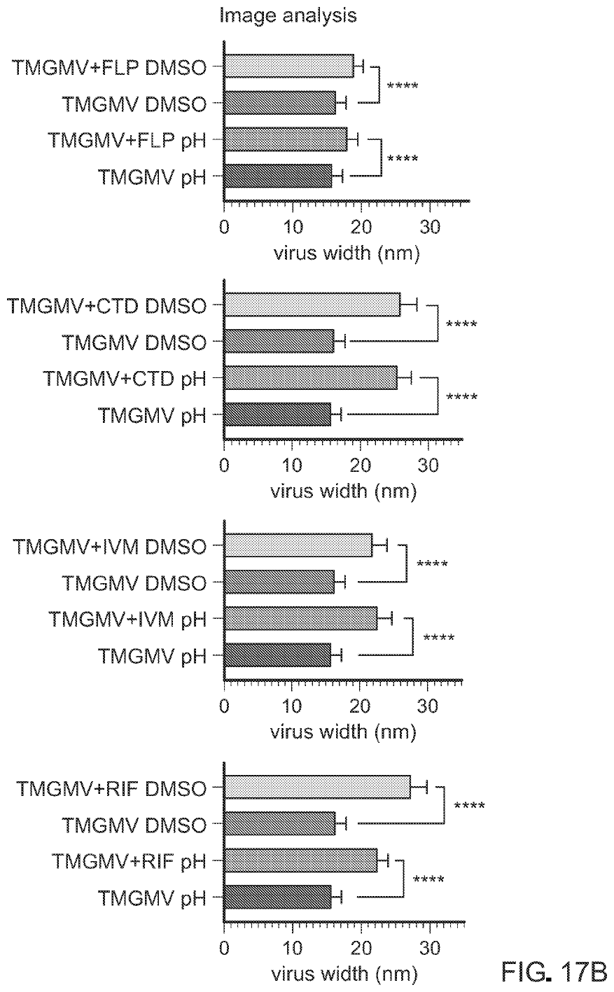

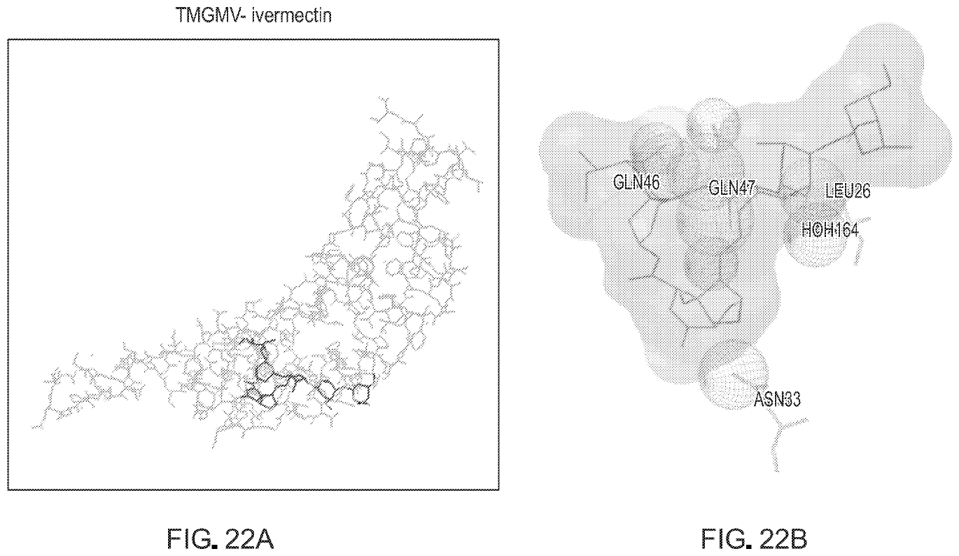

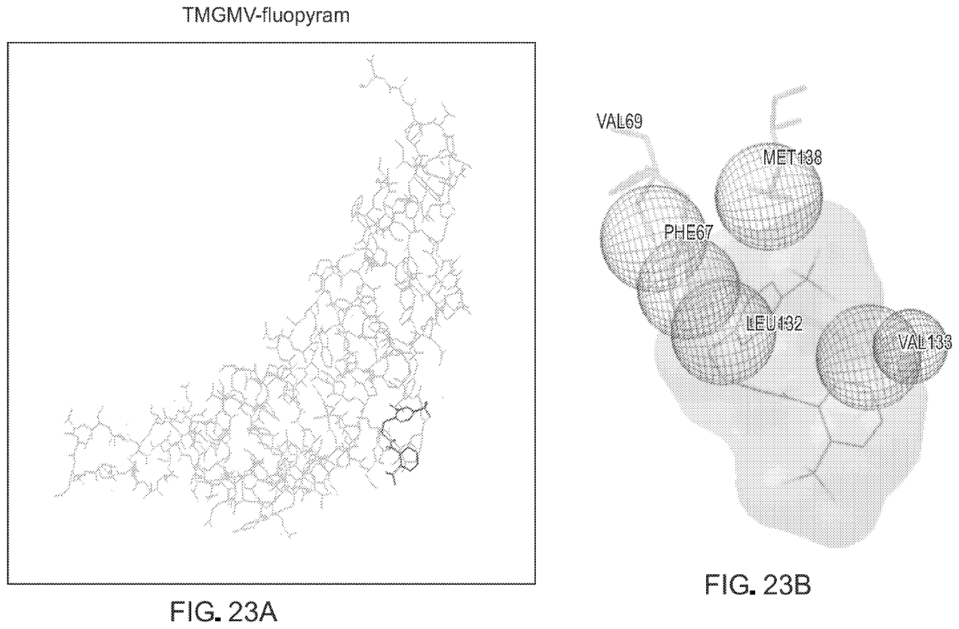

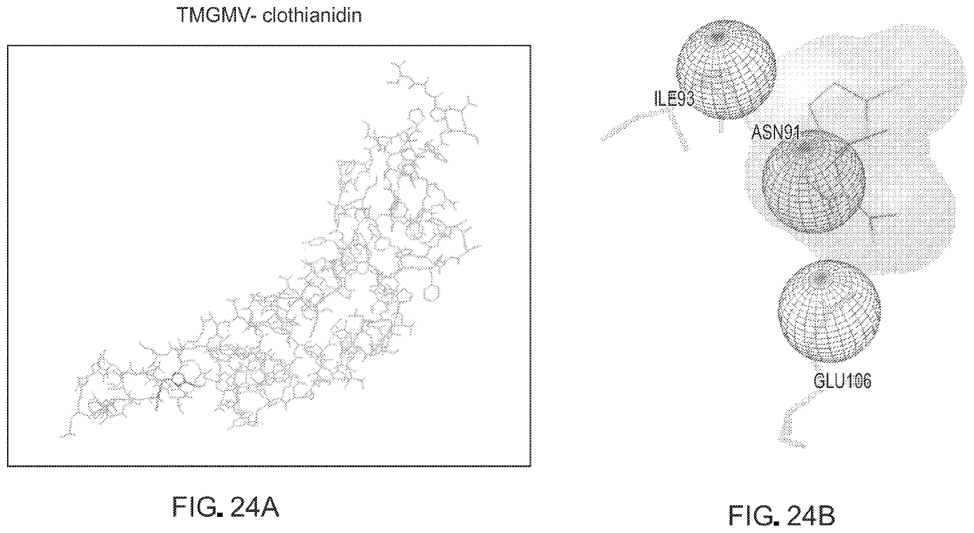

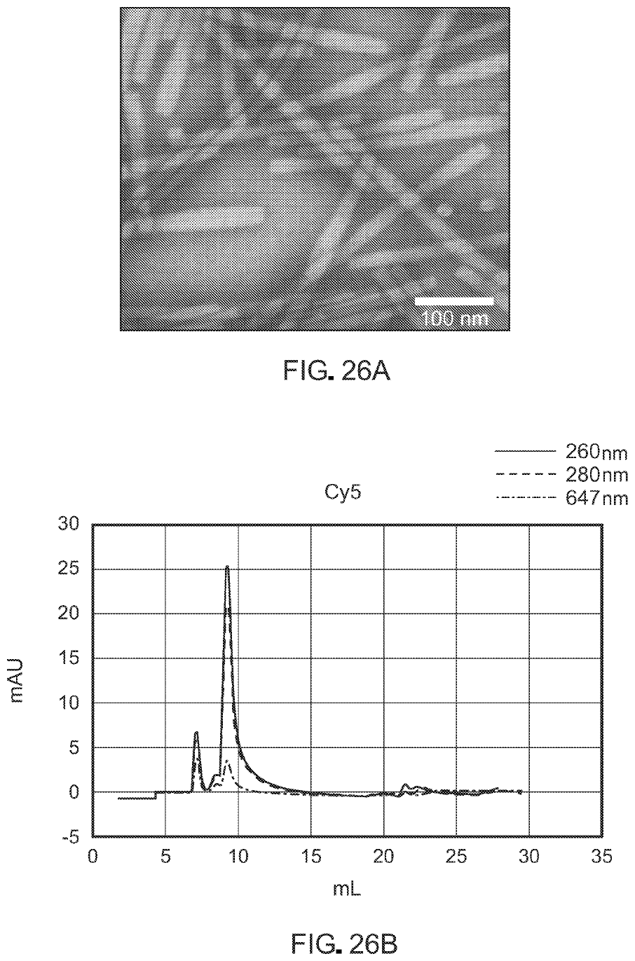

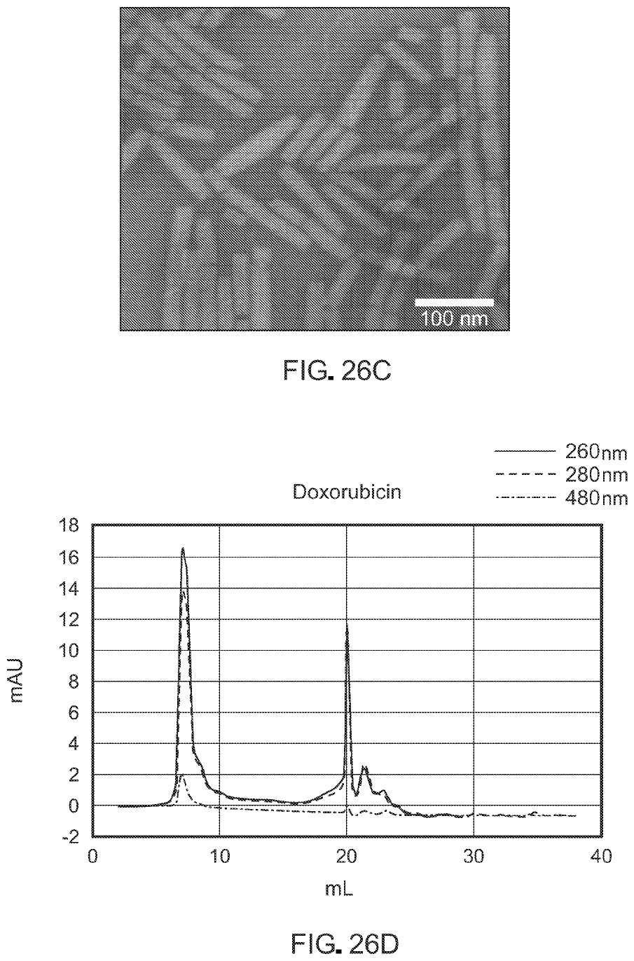

In some embodiments, the disclosure is directed to a “breathing” method for tobamovirus (e.g., TMGMV or TMV) based on careful pH adjustment (sometimes referred herein as the “pH method”) or concentration of a solvent (sometimes referred herein as the “solvent method”) and loaded molecules such as, but not limited to, fluopyram, clothianidin, rifampicin, and ivermectin (see, e.g., FIGS. 17-24). As shown in FIGS. 26A-26D, doxorubicin and cyanine5 (Cy5) were used as model active ingredients; the fluorescence of doxorubicin and Cy5 provides a convenient means for characterization. The nanoparticle formulations and tobamovirus (e.g., TMGMV or TMV) structures prepared via pH and solvent methods were characterized by a combination of techniques to determine particle integrity, AI infusion, and secondary structure stability post-infusion, as explained in Examples 5-10.

### Nanoparticles

In some embodiments, the nanoparticles of the disclosure are viral nanoparticles. In some embodiments, the viral nanoparticles are tobamoviruses. In some embodiments, the viral nanoparticles are tobacco mosaic viruses (TMV). In some embodiments, the viral nanoparticles are tobacco mild green mosaic viruses (TMGMV). In some embodiments, the viral nanoparticles are one or more species of the tobamovirus genus. In some embodiments, the viral nanoparticles are a bell pepper mottle virus (BPeMV), brugmansia mild mottle virus, cactus mild mottle virus (CMMoV), clitoria yellow mottle virus, cucumber fruit mottle mosaic virus, cucumber green mottle mosaic virus (CGMMV), cucumber mottle virus, frangipani mosaic virus (FrMV), hibiscus latent Fort Pierce virus (HLFPV), hibiscus latent Singapore virus (HLSV), kyuri green mottle mosaic virus, maracuja mosaic virus (MarMV), obuda pepper virus (ObPV), odontoglossum ringspot virus (ORSV), opuntia chlorotic ringspot virus, paprika mild mottle virus, passion fruit mosaic virus, pepper mild mottle virus (PMMoV), plumeria mosaic virus, rattail cactus necrosis-associated virus (RCNaV), rehmannia mosaic virus, ribgrass mosaic virus (HRV), sammons's Opuntia virus (SOV), streptocarpus flower break virus, sunn-hemp mosaic virus (SHMV), tobacco latent virus, tomato brown rugose fruit virus (ToBRFV), tomato mosaic virus (ToMV), tomato mottle mosaic virus, tropical soda apple mosaic virus, turnip vein-clearing virus (TVCV), ullucus mild mottle virus, wasabi mottle virus (WMoV), yellow tailflower mild mottle virus, youcai mosaic virus (YoMV) aka oilseed rape mosaic virus (ORMV), zucchini green mottle mosaic virus, or any combination thereof.

In some embodiments, the tobamovirus (e.g., TMGMV or TMV) is an engineered tobamovirus (e.g., TMGMV or TMV). In some embodiments, the engineered tobamovirus (e.g., TMGMV or TMV) is conjugated or otherwise linked to beta-cyclodextrin. In some embodiments, the beta-cyclodextrin is located on the outside surface of the tobamovirus (e.g., TMGMV or TMV). In some embodiments, the beta-cyclodextrin interacts with the active ingredient through supramolecular interactions. In some embodiments, the beta-cyclodextrin interacts with an active ingredient (AI) (e.g., a pesticide) through hydrophobic and/or hydrophilic interactions.

In some embodiments, the engineered tobamovirus (e.g., TMGMV or TMV) is modified to become infused with, impregnated with or otherwise contain an active ingredient (AI) (e.g., the tobamovirus (e.g., TMGMV or TMV) has been modified to “breathe” in the AI). In some embodiments, the tobamovirus (e.g., TMGMV or TMV) is partially and reversibly dissociated to allow for the incorporation of AI into the tobamovirus (e.g., TMGMV or TMV) structure. In some embodiments, one or more coat proteins of the tobamovirus (e.g., TMGMV or TMV) are reversibly and partially dissociated in response to an external factor. The external factor can be a change in pH or the presence of a solvent in a particular concentration. For example, changes in pH result in changes in the ionization of protein residues, which in turn affect electrostatic interactions between them. When the capsid proteins start to dissociate, the nanoparticle structure “breathes” and pores or pockets are opened allowing access to the inter-coat protein space. Besides pH, solvents also trigger assembly and disassembly. For example, in some embodiments, a solvent at concentrations of about 15% (v/v) to about 25% (v/v) tends to destabilize the proteins leading to dissociation and unfolding, due to its effects on charge state distribution, as well as disrupting structural water in the protein. In some embodiments, the solvent is a polar, aprotic solvent. In some embodiments, the solvent is a polar, aprotic solvent that is miscible in water. In some embodiments, the solvent is dimethylsulfoxide (DMSO).

In some embodiments, the one or more AIs are non-covalently conjugated to and entrapped within the one or more pores or pockets of the tobamovirus (e.g., TMGMV or TMV). In some embodiments, the one or more AIs are intercalated in the one or more coat proteins of the tobamovirus (e.g., TMGMV or TMV). In some embodiments, the one or more AIs are not chemically altered when loaded into the tobamovirus (e.g., TMGMV or TMV). In some embodiments, the impregnated tobamovirus (e.g., TMGMV or TMV) contains AI that is located within the tobamovirus (e.g., TMGMV or TMV). In some embodiments, the impregnated tobamovirus (e.g., TMGMV or TMV) contains AI that is dispersed within and throughout the tobamovirus (e.g., TMGMV or TMV). In some embodiments, the impregnated tobamovirus (e.g., TMGMV or TMV) does not interact with the AI on the surface of the tobamovirus (e.g., TMGMV or TMV). In some embodiments, the TMG tobamovirus (e.g., TMGMV or TMV) MV does not have an AI shell located on the outer surface of the tobamovirus (e.g., TMGMV or TMV).

In some embodiments, the AI-loaded tobamovirus (e.g., TMGMV or TMV) and non-loaded tobamovirus (e.g., TMGMV or TMV) are rod-shaped. In some embodiments, the engineered tobamovirus (e.g., TMGMV or TMV) has a different shape (e.g., different width) than a non-engineered tobamovirus (e.g., TMGMV or TMV). For example, upon loading the tobamovirus (e.g., TMGMV or TMV) with AI, the AI-loaded tobamovirus (e.g., TMGMV or TMV) can exhibit an increased width than a non-loaded tobamovirus (e.g., TMGMV or TMV) or a reference tobamovirus (e.g., TMGMV or TMV), as shown in FIG. 17A. In some embodiments, the AI-loaded tobamovirus (e.g., TMGMV or TMV) may appear swollen compared to a non-loaded tobamovirus (e.g., TMGMV or TMV) or a reference tobamovirus (e.g., TMGMV or TMV). In some embodiments, this change in width of the AI-loaded tobamovirus (e.g., TMGMV or TMV) suggest AI entrapment.

In some embodiments, an engineered tobamovirus (e.g., TMGMV or TMV)—AI nanoparticle is about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, or 105% larger than that of a non-engineered tobamovirus (e.g., TMGMV or TMV)—AI particle. In some embodiments, the width of an engineered tobamovirus (e.g., TMGMV or TMV)—AI nanoparticle is 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nm.

In some embodiments, the nanoparticles provided herein can be rod-shaped or can have an amorphous shape. In some embodiments, the nanoparticles provided herein have a length (extending between a first end and a second end of the exterior surface of the rod-shaped nanoparticle) ranging from about 2 nm to about 300 nm (e.g., about 2 nm to about 50 nm, about 2 nm to about 100 nm, about 2 nm to about 200 nm, about 2 nm to about 250 nm, about 2 nm to about 300 nm, about 50 nm to 75 nm, about 50 nm to about 100 nm, about 50 nm to about 125 nm, about 50 nm to about 150 nm, about 50 nm to about 175 nm, about 50 nm to about 200 nm, about 50 nm to about 225 nm, about 50 nm to about 250 nm, about 50 nm to about 275 nm, about 50 nm to about 300 nm, about 100 nm to about 125 nm, about 100 nm to about 150 nm, about 100 nm to about 175 nm, about 100 nm to about 200 nm, about 100 nm to about 225 nm, about 100 nm to about 250 nm, about 100 nm to about 275 nm, about 100 nm to about 300 nm, about 150 nm to about 175 nm, about 150 nm to about 200 nm, about 150 nm to about 225 nm, about 150 nm to about 250 nm, about 150 nm to about 275 nm, about 150 nm to about 300 nm, about 200 nm to about 225 nm, about 200 nm to about 250 nm, about 200 nm to about 275 nm, about 200 nm to about 300 nm, about 250 nm to about 275 nm, or about 250 nm to about 300 nm). In some embodiments, the nanoparticles provided herein have a length between about 50 nm to about 300 nm. In some embodiments, the nanoparticles provided herein have a length between about 50 nm. In some embodiments, the nanoparticles provided herein have a length between about 100 nm. In some embodiments, the nanoparticles provided herein have a length between about 150 nm. In some embodiments, the nanoparticles provided herein have a length between about 200 nm. In some embodiments, the nanoparticles provided herein have a length between about 250 nm. In some embodiments, the nanoparticles provided herein have a length between about 300 nm.

In some embodiments, the nanoparticle of the disclosure comprises about 1 to about 2000 AI molecules per TMGMV or more (e.g., about 1 to 10, 1 to 15, 1 to 20, 1 to 50, 1 to 60, 1 to 75, 1 to 100, 1 to 150, 1 to 175, 1 to 185, 1 to 200, 1 to 300, 1 to 400, 1 to 500, 1 to 600, 1 to 700, 1 to 800, 1 to 900, 1 to 1000, 1 to 1100, 1 to 1500, 1 to 1999, 10 to 15, 10 to 20, 10 to 50, 10 to 60, 10 to 75, 10 to 100, 10 to 150, 10 to 175, 10 to 185, 10 to 200, 10 to 300, 10 to 400, 10 to 500, 10 to 600, 10 to 700, 10 to 800, 10 to 900, 10 to 1000, 10 to 1100, 10 to 1500, 10 to 2000, 50 to 60, 50 to 75, 50 to 100, 50 to 150, 50 to 175, 50 to 185, 50 to 200, 50 to 300, 50 to 400, 50 to 500, 50 to 600, 50 to 700, 50 to 800, 50 to 900, 50 to 1000, 50 to 1100, 50 to 1500, 50 to 2000, 150 to 175, 150 to 185, 150 to 200, 150 to 300, 150 to 400, 150 to 500, 150 to 600, 150 to 700, 150 to 800, 150 to 900, 150 to 1000, 150 to 1100, 150 to 1500, 150 to 2000, 500 to 600, 500 to 700, 500 to 800, 500 to 900, 500 to 1000, 500 to 1100, 500 to 1500, 500 to 2000, 750 to 800, 750 to 900, 750 to 1000, 750 to 1100, 750 to 1500, 750 to 2000, 1000 to 1100, 1000 to 1500, 1000 to 2000 AI molecules per tobamovirus (e.g., TMGMV or TMV), or more).

### Compositions

In some embodiments, the compositions of the disclosure contain a plurality of nanoparticles of the disclosure. In some embodiment, the plurality of nanoparticles comprise nanoparticles with the same engineered modifications (e.g., a population of engineered tobamovirus, wherein the population of tobamovirus is linked to beta-cyclodextrin, or a population of engineered tobamovirus, wherein the population is impregnated with a particular AI). In some embodiments, the plurality of nanoparticles includes nanoparticles carrying the same AI. In some embodiments, the composition comprises a mixture of nanoparticles, wherein some of the nanoparticles of the mixture comprise beta-cyclodextrin (BCD) linked tobamovirus, and some of the nanoparticles of the mixture comprise impregnated tobamovirus. In some embodiments, the composition comprises a mixture of nanoparticles, wherein the nanoparticles carry or are impregnated with different AIs (e.g., pesticides). For example, a composition of the disclosure can comprise a mixture of nanoparticles wherein nanoparticle “A” comprised of a plurality of BCD-linked tobamovirus carrying pesticide “1” is mixed with nanoparticle “B” comprised of a plurality of tobamovirus impregnated with pesticide “1”. Other non-limiting examples include wherein nanoparticle “A” comprised of a plurality of BCD-linked tobamovirus carrying pesticide “1” is mixed with nanoparticle “B” comprised of a plurality of BCD-linked tobamovirus carrying pesticide “2”, or wherein nanoparticle “A” comprised of a plurality of tobamovirus impregnated with pesticide “1” is mixed with nanoparticle “B” comprised of a plurality of tobamovirus impregnated with pesticide “2”. In some embodiments, mixtures with three or more, four or more, or five or more pluralities of nanoparticles of the disclosure are also included.

In some embodiments, the disclosed nanoparticles can be formulated into an aqueous solution. In some embodiments, the nanoparticles can be formulated into hydrogel. In some embodiments, the disclosed nanoparticles can be lyophilized. In some embodiments, the disclosed nanoparticles are formulated into re-dispersible powders and aqueous dispersions. In some embodiments, the nanoparticles comprise high weight percentages of a water-insoluble pesticide or other active ingredient. In some embodiments, the disclosed nanoparticles can be prepared from an oil-in-water microemulsion or nanoemulsion containing a water insoluble, non-halogenated volatile organic solvent, from which organic solvent and/or water has been removed.

In some embodiments, a composition comprising any of the disclosed nanoparticles can also comprise a solvent. Examples of a solvent include, but are not limited to, dimethylsulfoxide (DMSO), ethanol, 1-propanol, 2-propanol, n-pentanol, n-butanol, ethyl acetate, tetrahydrofuran, propylene glycol, formamide, glycerol, polyethylene glycol and mixtures thereof. In another embodiment, the co-solvent is present in an amount of about 5 to about 30% by weight based on the total weight of the microemulsion.

In some embodiments, the disclosed nanoparticles are in liquid formulations. In some embodiments, the liquid formulation comprises an AI and a disclosed nanoparticle, which is dissolved, emulsified as droplets or suspended as matrix particles. In some embodiments, the liquid formulation comprises a disclosed nanoparticle and an AI such as a pesticide, which is dissolved or emulsified as droplets. In some embodiments, the pesticide is homogenously distributed throughout the particle.

In some embodiments, any nanoparticle formulation described herein can be used as such or use forms prepared there from, for example in the form of directly sprayable solutions, powders, suspensions or dispersions, emulsions, oil dispersions, pastes, dustable products, materials for spreading, or granules, by means of spraying, atomizing, dusting, spreading or pouring. In some embodiments, the use forms depend entirely on the intended purposes; for example, a formulation is intended to ensure in each case the finest possible distribution of the pesticid(es) and nanoparticle described herein.

In some embodiments, aqueous use forms can be prepared also from emulsion concentrates, pastes or wettable powders (sprayable powders, oil dispersions) by adding a suitable solvent, for example water. In some embodiments, a disclosed nanoparticle can be used individually or already partially or completely mixed with one another and/or any AI disclosed herein to prepare the composition according to the disclosure.

In some embodiments, a composition comprising any of the disclosed nanoparticles can also comprise a surfactant or a mixture of surfactants. In one embodiment, the surfactant is any one or more of a cationic surfactant, an anionic surfactant, an amphoteric surfactant, a nonionic surfactant and mixtures thereof. In some embodiments, the anionic surfactant is selected from the group consisting of an alkyl benzene sulfonate (e.g., sodium alkyl naphthalene sulfonate), sodium dodecyl sulfate, sodium sulfosuccinate, sodium lauryl sulfate, alkyl naphthalene sulfonate condensate sodium salt, sodium stearate, and mixtures thereof; the nonionic surfactant is selected from the group consisting of an ethoxylated sorbitan ester, a sorbitan ester, an organosilicone surfactant, a polyglycerol ester, a sucrose ester, a poloxamer, an alkyl polyglucoside, polyalkyleneoxide modified heptamethyltrisiloxanes, and allyloxypolyethylene glycol methylether and mixtures thereof; the amphoteric surfactant is lecithin; and the cationic surfactant is selected from the group consisting of cetyl trimethyl ammonium bromide, cetyl trimethyl ammonium chloride, and mixtures thereof. In some embodiments, the surfactant is present in an amount of about 5 to about 35% by weight based on the total weight of the microemulsion. In some embodiments, the surfactant is Morwet® (sodium n-butyl naphthalene sulfonate). In some embodiments, the surfactant is Silwet® L-77 (an organosilicone surfactant comprising a blend of polyalkyleneoxide modified heptamethyltrisiloxane and allyloxypolyethylene glycol methyl ether.

In some embodiments, the nanoparticles of the disclosure can be formulated within a matrix. As used herein, the term “matrix” has its ordinary meaning and refers to a mixture in which the nanoparticles are suspended throughout another substance. Thus, in some embodiments, the composition includes dispersed or suspended nanoparticles. In the scope of the disclosure, a matrix fluid refers to a composition that has been activated using any one or more of the activation means. In some embodiments, a matrix is a hydrogel.

In some embodiments, the compositions of the disclosure comprise an excipient. In some embodiments, the excipient is a buffer or water. In some embodiments, the buffer is potassium phosphate buffer. In some embodiments, the water is deionized water.

### Active Ingredients

Any nanoparticle described herein can at least include one active ingredient (AI) or one or more AIs. In some embodiments, at least one AI comprises at least one of a drug, pesticide, or a small molecule. In some embodiments, the drug can be a chemokine, an antibacterial, or any therapeutic compound. In some embodiments, the drug is a chemotherapeutic drug, an antiparasitic drug, an antibiotic, an immunomodulator, an anti-fungal drug, an anti-protozoal drug, an antiviral drug, or any combination thereof. In some embodiments, the drug is a hydrophilic drug or a hydrophobic drug.

In some embodiments, the chemotherapeutic agent is a small molecule, a protein (e.g., an antibody, an antigen-binding fragment of an antibody, or a derivative or conjugate thereof), a nucleic acid, or any combination thereof. Non-limiting examples of chemotherapeutic agents include: cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, azacitidine, axathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, vinorelbine, and bevacizumab (or an antigen-binding fragment thereof).

In some embodiments, the antiparasitic drug is niclosamide, oxyclozanide, rafoxanide, closantel, dibromsalan, metabromsalan, tribromsalan and nitazoxanide.

In some embodiments, the antibiotic is a beta-lactam antibiotic, aminoglycoside, ansa-type antibiotic, anthraquinone, antibiotic azole, antibiotic glycopeptide, macrolide, antibiotic nucleoside, antibiotic peptide, antibiotic polyene, antibiotic polyether, quinolone, antibiotic steroid, sulfonamide, tetracycline, dicarboxylic acid, antibiotic metal, oxidizing agent, a substance that releases free radicals and/or active oxygen, cationic antimicrobial agent, quaternary ammonium compound, biguanide, triguanide, bisbiguanide and analogs and polymers thereof, naturally occurring antibiotic compound, and any combination thereof. In some embodiments, the AI is rifampicin. In some embodiments, the AI is ivermectin. In some embodiments, the AI is fluopyram. In some embodiments, the AI is clothianidin.

In some embodiments, the immunomodulator is a substance that stimulates or suppresses the immune system and may help the body fight cancer, infection, or other diseases. In some embodiments, the immunomodulator is a cancer immunotherapeutic, such as but not limited to, checkpoint inhibitors, adoptive cell therapy (T-cell transfer therapy, monoclonal antibody therapy, cancer vaccines, immune system modulators (e.g., cytokines and biologic response modifiers such as thalidomide, lenalidomide, pomalidomide, and imiquimod), or any combination thereof. In some embodiments, the immunomodulator is a corticosteroid, a disease-modifying antirheumatic drug (DMARD) (e.g., azathioprine, cyclosporine, hydroxychloroquine, leflunomide, methotrexate, and sulfasalazine), biologics (e.g., tumor necrosis factor (TNF) inhibitors, interleukin-1 (IL-1) inhibitors, interleukin-6 (IL-6) inhibitors, T-cell inhibitor, B-cell inhibitor), Janus kinase inhibitors, or any combination thereof. In some embodiments, the immunomodulator is GM-CSF (granulocyte-macrophage colony stimulating factor).

Any nanoparticle described herein can at least one active ingredient (AI) or one or more AIs. In some embodiments, at least one AI comprises at least one of a drug, pesticide, or a small molecule. In some embodiments, the drug can be a chemokine, an antibacterial, or any therapeutic compound. In some embodiments, the drug is a chemotherapeutic drug, an antiparasitic drug, an antibiotic drug, or an immunomodulator. In some embodiments, the drug is a hydrophilic drug or a hydrophobic drug. In some embodiments, the pesticide is a water-insoluble organic compound, an insecticide, a herbicide, a fungicide, an acaricide, an algicide, an antimicrobial agent, biopesticide, a biocide, a disinfectant, a fumigant, an insect growth regulator, a plant growth regulator, a miticide, a microbial pesticide, a molluscide, a nematicide, an ovicide, a pheromone, a repellent, a rodenticide, a defoliant, a desiccant, a safener, or any combination thereof. In some embodiments, the pesticide is a benzoyl urea, such as novaluron, lufenuron, chlorfluazuron, flufenoxuron, hexaflumuron, noviflumuron, teflubenzuron, triflumuron and diflubenzuron; a carbamate; a pyrethroid, such as cyhalothrin and isomers and isomer mixtures thereof, lambda-cyhalothrin, deltamethrin, tau-fluvalinate, cyfluthrin, beta-cyfluthrin, tefluthrin, and bifenthrin; an organophosphate, such as azinfos-methyl, chlorpyrifos, diazinon, endosulfan, methidathion; a neonicotinoid; a phenylpyrazole, such as imidacloprid, acetamiprid, thiacloprid, dinotefuran, thiamethoxam, and fipronil; a conazole, such as epoxiconazole, hexaconazole, propiconazole, prochloraz, imazalil, triadimenol, difenoconazole, myclobutanil, prothioconazole, triticonazole, and tebuconazole; a morpholine, such as dimethomorph, fenpropidine, and fenpropimorph; a strobilurin, such as azoxystrobin, kresoxim-methyl, and analogues thereof; a phthalonitrile, such as chlorothalonil; a mancozeb; a fluazinam; a pyrimidine, such as bupirimate; an aryloxyphenoxy derivative; an aryl urea; an aryl carboxylic acid; an aryloxy alkanoic acid derivative, such as clodinafop-propargyl and analogues thereof, fenoxaprop-p-ethyl and analogues thereof, propaquizafop, quizalafop and analogues thereof; a dintroaniline, such as pendimethalin and trifluralin; a diphenyl ether, such as oxyfluorfen; an imidazolinone; a sulfonylurea, such as chlorsulfuron, nicosulfuron, rimsulfuron, tribenuron-methyl; a sulfonamide; a triazine; and a triazinone, such as metamitron; and any combination thereof. In some embodiments, any nanoparticle described herein can comprise one or more compounds selected from the group consisting of fungicides, insecticides, nematicides, herbicide and/or safener or growth regulator. Any pesticides of two or more the aforementioned classes can be used. The skilled artisan is familiar with useful drugs and pesticides, which can be, for example, found in the Pesticide Manual, 13th Ed. (2003), The British Crop Protection Council, London.

Any nanoparticle, composition or method described herein can comprise any one or more of the following list of pesticides, which is intended to illustrate the possible combinations, but not to impose any limitation:
A) a strobilurin,
azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)-phenyl)-2-methoxyimino-N-methyl-acetamide, 3-methoxy-2-(2-(N-(4-methoxy-phenyl)-cyclopropane-carboximidoylsulfanylmethyl)-phenyl)-acrylic acid methyl ester, methyl(2-chloro-5-[1-(3-methylbenzyloxyimino)ethyl]benzyl)carbamate and 2-(2-(3-(2,6-dichlorophenyl)-1-methyl-allylideneaminooxymethyl)-phenyl)-2-methoxyimino-N-methyl-acetamide;
B) a carboxamide,
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, tecloftalam, thifluzamide, tiadinil, 2-amino-4-methyl-thiazole-5-carboxanilide, 2-chloro-N-(1,1,3-trimethyl-indan-4-yl)-nicotinamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-3-difluoromethyl-1-methyl-1H-pyrazole-4-carboxamide. N-(2′,5′-dichlorobiphenyl-2-yl)-3-difluoromethyl-1-methyl-1H-pyrazole-4-carboxamide, N-(3′,5′-difluorobiphenyl-2-yl)-3-difluoromethyl-1-methyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′,4′,5′-trifluorobiphenyl-2-yl)-3-difluoromethyl-1-methyl-1H-pyrazole-4-carboxamide, N-(2′,4′,5′-trifluorobiphenyl-2-yl)-3-difluoromethyl-1-methyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[2-(1,1,2,2-tetrafluoroethoxy)-phenyl]-3-difluoromethyl-1-methyl-1H-pyrazole-4-carboxamide, N-(4′-trifluoromethyl-thiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide, N-(2-(1,3,3-trimethyl-butyl)-phenyl)-1,3-dimethyl-5-fluoro-1H-pyrazole-4-carboxamide, N-(4′-chloro-3′,5′-difluoro-biphenyl-2-yl)-3-difluoromethyl-1-methyl-1H-pyrazole-4-carboxamide, N-(4′-chloro-3′,5′-difluoro-biphenyl-2-yl)-3-trifluoromethyl-1-methyl-1H-pyrazole-4-carboxamide, N-(3′,4′-dichloro-5′-fluoro-biphenyl-2-yl)-3-trifluoromethyl-1-methyl-1H-pyrazole-4-carboxamide, N-(3′,5′-difluoro-4′-methyl-biphenyl-2-yl)-3-difluoromethyl-1-methyl-1H-pyrazole-4-carboxamide, N-(3′,5′-difluoro-4′-methyl-biphenyl-2-yl)-3-trifluoromethyl-1-methyl-1H-pyrazole-4-carboxamide, N-(2-bicyclopropyl-2-yl-phenyl)-3-difluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoromethyl-1-methyl-1H-pyrazole-4-carboxamide, N-[1,2,3,4-tetrahydro-9-(1-methylethyl)-1,4-methano-naphthalen-5-yl]-3-(difluoromethyl)-1-methyl-TH-pyrazole-4-carboxamide;
carboxylic morpholides: dimethomorph, flumorph;
benzoic acid amides: flumetover, fluopicolde, fluopyram;
other carboxamides: carpropamid, dicyclomet, mandiproamid, oxytetracyclin, silthiofarm and N-(6-methoxy-pyridin-3-yl)cyclopropanecarboxylic acid amide;
C) an azole,
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([1,2,4]triazol-1-yl)-cycloheptanol;
imidazoles: cyazofamid, imazalil, pefurazoate, prochloraz, triflumizol;
benzimidazoles: benomyl, carbendazim, fuberidazole, thiabendazole;
others: ethaboxam, etridiazole, hymexazole and 2-(4-chloro-phenyl)-N-[4-(3,4-dimethoxy-phenyl)-isoxazol-5-yl]-2-prop-2-ynyloxy-acetamide;
D) a heterocyclic compound,
pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fenpiclonil, fludioxonil;
morpholines: aldimorph, dodemorph, dodemorph-acetate, fenpropimorph, tridemorph;
piperidines: fenpropidin;
dicarboximides: fluoroimid, iprodione, procymidone, vinclozolin;
non-aromatic 5-membered heterocycles: famoxadone, fenamidone, octhilinone, probenazole, 5-amino-2-isopropyl-3-oxo-4-ortho-tolyl-2,3-dihydro-pyrazole-1-carbothioic acid S-allyl ester;
and/or any others: acibenzolar-S-methyl, amisulbrom, anilazin, blasticidin-S, captafol, captan, chinomethionat, dazomet, debacarb, diclomezine, difenzoquat, difenzoquat-methyl-sulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, 5-chloro-7-(4-methyl-piperidin-1-yl)-6-(2,4,6-trifluorophenyl)-[1,2,4]triazolo[1,5-a]pyrimidine, 6-(3,4-dichloro-phenyl)-5-methyl-[1,2,4]triazolo[1,5-a]pyrimidine-7-ylamine, 6-(4-tert-butyl-phenyl)-5-methyl-[1,2,4]triazolo[1,5-a]pyrimidine-7-ylamine, 5-methyl-6-(3,5,5-trimethyl-hexyl)-[1,2,4]triazolo[1,5-a]pyrimidine-7-ylamine, 5-methyl-6-octyl-[1,2,4]triazolo[1,5-a]pyrimidine-7-ylamine, 6-methyl-5-octyl-[1,2,4]triazolo[1,5-a]pyrimidine-7-ylamine, 6-ethyl-5-octyl-[1,2,4]triazolo[1,5-a]pyrimidine-7-ylamine, 5-ethyl-6-octyl-[1,2,4]triazolo[1,5-a]pyrimidine-7-ylamine, 5-ethyl-6-(3,5,5-trimethyl-hexyl)-[1,2,4]triazolo[1,5-a]pyrimidine-7-ylamine, 6-octyl-5-propyl-[1,2,4]triazolo[1,5-a]pyrimidine-7-ylamine, 5-methoxymethyl-6-octyl-[1,2,4]triazolo[1,5-a]pyrimidine-7-ylamine, 6-octyl-5-trifluoromethyl-[1,2,4]triazolo[1,5-a]pyrimidine-7-ylamine and 5-trifluoromethyl-6-(3,5,5-trimethyl-hexyl)-[1,2,4]triazolo[1,5-a]pyrimidine-7-ylamine;
E) a carbamate,
thio- and dithiocarbamates: ferbam, mancozeb, maneb, metam, methasulphocarb, metiram, propineb, thiram, zineb, ziram;
carbamates: benthiavalicarb, diethofencarb, flubenthiavalicarb, iprovalicarb, propamocarb, propamocarb hydrochlorid, valiphenal and N-(1-(1-(4-cyano-phenyl)-ethanesulfonyl)-but-2-yl)carbamic acid-(4-fluorophenyl)ester;
F) any other active substances,
guanidines: guanidine, dodine, dodine free base, guazatine, guazatine-acetate, iminoctadine, iminoctadine-triacetate, iminoctadine-tris(albesilate);
antibiotics: kasugamycin, kasugamycin hydrochloride-hydrate, streptomycin, polyoxine, validamycin A;
nitrophenyl derivates: binapacryl, dinobuton, dinocap, nitrthal-isopropyl, teenazen, organometal compounds: fentin salts, such as fentin-acetate, fentin chloride or fentin hydroxide;
sulfur-containing heterocyclyl compounds: dithianon, isoprothiolane;
organophosphorus compounds: edifenphos, fosetyl, fosetyl-aluminum, iprobenfos, phosphorus acid and its salts, pyrazophos, tolclofos-methyl;
organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide;
an inorganic active substance: Bordeaux mixture, copper acetate, copper hydroxide, copper oxychloride, basic copper sulfate, sulfur;
any one or more of others: biphenyl, bronopol, cyflufenamid, cymoxanil, diphenylamin, metrafenone, mildiomycin, oxin-copper, prohexadione-calcium, spiroxamine, tolylfluanid, N-(cyclopropylmethoxyimino-(6-difluoro-methoxy-2,3-difluoro-phenyl)-methyl)-2-phenyl acetamide, N′-(4-(4-chloro-3-trifluoromethvl-phenoxy)-2,5-dimethyl-phenyl)-N-ethyl-N-methyl formamidine, N′-(4-(4-fluoro-3-trifluoromethyl-phenoxy)-2,5-dimethyl-formamidine, N′-(2-methyl-5-trifluoromethyl-4-(3-trimethylsilanyl-propoxy)-phenyl)-N-ethyl-N-methyl formamidine and N′-(5-difluoromethyl-2-methyl-4-(3-trimethvlsilanyl-propoxy)-phenyl)-N-ethyl-N-methyl formamidine.
G) an herbicide such as an acetamide: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid derivatives: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
Bipyridyls: diquat, paraquat;
(thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bomoxynil, dichlobenil, ioxynil;
imidazolinones: imazamethabenz, imazamox, imazapic, imazapyr, imazaquin, imazethapyr;
phenoxy acetic acids: clomeprop, 2,4-dichlorophenoxyacetic acid (2,4-D), 2,4-DB, dichlorprop, MCPA, MCPA-thioethyl, MCPB, Mecoprop;
pyrazines: chloridazon, flufenpyr-ethyl, fluthiacet, norflurazon, pyridate; pyridines: aminopyralid, clopyralid, diflufenican, dithiopyr, fluridone, fluroxypyr, picloram, picolinafen, thiazopyr;
sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;
triazines: ametryn, atrazine, cyanazine, dimethametryn, ethiozin, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamiine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester;
H) any one or more of an insecticide, which can be selected from the groups consisting of
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
I) any one or more of an insect growth regulator: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
J) any one or more of any other compounds such as:
nicotinic receptor agonists/antagonists compounds: clothianidin, dinotefuran, imidacloprid, thiamethoxam, nitenpyram, acetamiprid, thiacloprid, 1-(2-chloro-thiazol-5-ylmethyl)-2-nitrimino-3,5-dimethyl-[1,3,5]triazinane;
GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;
macrocyclic lactone insecticides: abamectin, emamectin, milbemectin, lepimectin, spinosad, spinetoram;
mitochondrial electron transport inhibitor (METI) I acaricides: fenazaquin, pyridaben, tebufenpyrad, tolfenpyrad, flufenerim;
METI II and III compounds: acequinocyl, fluacyprim, hydramethylnon;
Uncouplers: chlorfenapyr;
oxidative phosphorylation inhibitors: cyhexatin, diafenthiuron, fenbutatin oxide, propargite;
moulting disruptor compounds: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, and pyrifluquinazon;
K) the growth regulator can be selected from any one or more of abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole.

In some embodiments, the pesticide is sensitive to UV light. The sensitivity may be detected by simple tests, in which a pesticide is exposed to UV light for a certain time. Subsequently, residual pesticide, which was not decomposed, may be quantified.

In some embodiments the nanoparticles comprise herbicides such as napropamid, proparil, Bentazone, Paraquat dichlorid, cycloxydim, sethoxydim, Ethalfluralin, Oryzalin, Pendimethalin, Trifluralin, Acifluren, Aclonifen, Fomesafen, oxyfluoren, Ioxynil, Imazetapyr, Imazaquin, chloridazon, norflurazon, Thiazopyr, Triclopyr, dithiopyr, Diflufenican, picolinafen, amidosulfuron, Molinate, vernolate, Promethon, Metribuzin, azafenidin, Carfentrazone-ethyl, sulfentrazone, metoxuron, monolinuron, Fluchloralin and Flurenol.

In some embodiments the nanoparticles comprise fungicides such as cyprodinil, Fuberidazol, dimethomorph, procloraz, Triflumizol, tridemorph, edifenfos, Fenarimol, Nuarimol, ethirimol, quinoxylen, Dithianon, Metominostrobin, Trifloxystrobin, Dichlofluamid, Bromuconnazol and myclobutanil.

In some embodiments the nanoparticles comprise insecticides such as Acephate, Azinphos-Ethyl, Azinphos-Methyl, Isofenphos, Chlorpyriphos-Methyl, Dimethylvinphos, Phorate, Phoxim, Prothiofos, cyhexatin, alanycarb, Ethiofencarb, pirimicarb, Thiodicarb, Fipronil, bioallethrin, bioresmethin, Deltamethrin, fenpropathin, Flucythrinate, Tau fluvalinate, cypermethrin, Zeta cypermethrin, resmethin, tefluthrin, Lambda cyhalothrin and hydramethylnon. In another preferred embodiment, the insecticide is metaflumizone or alpha-cypermethrin.

In some embodiments the nanoparticles comprise metaflumizone or alpha-cypermethrin.

### Methods of Making Nanoparticles

In certain embodiments, the disclosure is directed to a method of making a nanoparticle comprising tobamovirus (e.g., TMGMV and/or TMV) and one or more active ingredients (AIs) comprising an adjustment of the pH of the solution the nanoparticles are suspended in (e.g., the “pH method”). In some embodiments, the method includes the steps of providing isolated tobamovirus (e.g., TMGMV and/or TMV) to a buffer with a pH of about 7 to 9 to create a tobamovirus-buffer, adding one or more AIs to the tobamovirus-buffer more than once, thereby creating the nanoparticle, and purifying the nanoparticle in a solution with a pH of about 5 to 9. This method is further described in Example 5. In some embodiments, the one or more AIs are non-covalently conjugated to the tobamovirus (e.g., TMGMV and/or TMV). In some embodiments, the tobamovirus (e.g., TMGMV and/or TMV) comprises one or more coat proteins that reversibly and partially dissociate in response to a change in pH.

In some embodiments, the one or more AIs are added at least once a day for at least 1, 2, 3, 4, 5, 6, 7, 8, 9, 10, 11, 12, 13, 14, 15, 16, 17, 18, 19, 20, or 21 days. In some embodiments, the pH of the tobamovirus-buffer is about 7 to 7.5, 7.5 to 8, 7 to 8, 8 to 8.5, 8.5 to 9, or 8 to 9. In some embodiments, the pH of the tobamovirus-buffer is about 7.2 to 7.8, 7.3 to 7.8, 7.2 to 7.7, 7.3 to 7.7, 7.4 to 7.8, 7.4 to 7.7, 7.5 to 7.7, 7.5 to 7.8, 7.2 to 7.6, 7.3 to 7.6, 7.4 to 7.6, 7.5 to 7.6, 7.2 to 7.5, 7.3 to 7.5, 7.4 to 7.5, 7.2 to 7.9, 7.3 to 7.9, 7.4 to 7.9, 7.5 to 7.9, 7.3 to 7.99, 7.4 to 7.99, or 7.5 to 7.99. In some embodiments, the pH of the tobamovirus-buffer is about 7.2, 7.3, 7.4, 7.5, 7.6, 7.7, 7.8, 7.9, or 7.99. In some embodiments, the pH of the tobamovirus-buffer is 7.5.

In some embodiments, the solution in which the nanoparticles are purified in 30 has a pH of about 5 to about 9 (e.g., about 5 to 6, 5 to 7, 5 to 8, 5 to 8.9, 6 to 7, 6 to 8, 6 to 9, 7 to 8, 7 to 9, 8 to 9, 5.5 to 6.5, 5.5 to 7.5, 5.5 to 8.5, 5.5 to 8.9, 6.5 to 7.5, 6.5 to 8.5, 6.5 to 8.9, 7.5 to 8.5, 7.5 to 9, 8.5 to 9, 6.9 to 7.1, 6.9 to 7.2, 6.9 to 7.3, 5 to 6.5, 5 to 7.5, 5 to 8.5, 5 to 8.9, 6 to 7.5, 6 to 8.5, 6 to 9.5, 7 to 7.1, 7 to 7.2, 7 to 7.3, 7 to 7.4, 7 to 7.5, 7 to 7.6, 7 to 7.7, 7 to 7.8, 7 to 7.9, 7 to 9.5, 8 to 9.5, 5.5 to 6, 5.5 to 7, 5.5 to 8, 5.5 to 8.9, 6.5 to 7, 6.5 to 8, 6.5 to 9, or 7.5 to 8). In some embodiments, the solution in which the nanoparticles are purified in has a pH of about 6.9, 7.0, 7.1, 7.2, or 7.3.

As described elsewhere herein, the change in pH results in a phase transition of the tobamovirus (e.g., TMGMV and/or TMV), thereby opening up “pores” or “pockets” that can receive and non-covalently link one or more AI molecules within. In some embodiments, the change in pH (e.g., the difference in pH of the tobamovirus-buffer versus the solution in which the nanoparticles are purified in) is about 0.5 to 1, about 0.5 to 2, 0.5 to 3, 1 to 2, or 1 to 3.

In certain embodiments, the disclosure is directed to a method of making a nanoparticle comprising tobamovirus (e.g., TMGMV and/or TMV) and one or more active ingredients (AIs) comprising an adjustment of the concentration of a solvent (e.g., DMSO) present in the solution that the nanoparticles are suspended in (e.g., this method is sometimes referred to as the “solvent method” throughout the disclosure). In some embodiments, the method includes the steps of providing isolated tobamovirus (e.g., TMGMV and/or TMV) to a buffer with a pH of about 5 to 9 to create a tobamovirus-buffer, adding a solvent at a concentration of about 15% (v/v) to about 30% (v/v), adding one or more AIs to the tobamovirus-buffer, thereby creating the nanoparticle, and purifying the nanoparticle in a solution with a pH of about 5 to 9.

In some embodiments, the step of providing the isolated tobamovirus includes using a buffer having a pH of about 5 to 6, 5 to 7, 5 to 8, 5 to 8.9, 6 to 7, 6 to 8, 6 to 9, 7 to 8, 7 to 9, 8 to 9, 5.5 to 6.5, 5.5 to 7.5, 5.5 to 8.5, 5.5 to 8.9, 6.5 to 7.5, 6.5 to 8.5, 6.5 to 8.9, 7.5 to 8.5, 7.5 to 9, 8.5 to 9, 6.9 to 7.1, 6.9 to 7.2, 6.9 to 7.3, 5 to 6.5, 5 to 7.5, 5 to 8.5, 5 to 8.9, 6 to 7.5, 6 to 8.5, 6 to 9.5, 7 to 7.1, 7 to 7.2, 7 to 7.3, 7 to 7.4, 7 to 7.5, 7 to 7.6, 7 to 7.7, 7 to 7.8, 7 to 7.9, 7 to 9.5, 8 to 9.5, 5.5 to 6, 5.5 to 7, 5.5 to 8, 5.5 to 8.9, 6.5 to 7, 6.5 to 8, 6.5 to 9, or 7.5 to 8, to create the tobamovirus-buffer. In some embodiments, the buffer has a pH of about 7, 7.1, 7.2, 7.3, 7.4, or 7.5.

In some embodiments, the solvent is a polar, aprotic solvent. In some embodiments, the solvent is a polar, aprotic solvent that is miscible with water. In some embodiments, the solvent is dimethylsulfoxide (DMSO). In some embodiments, the solvent is acetone, acetonitrile, dichloromethane, dimethylformamide, dimethylpropyleneurea, dimethylsulfoxide, ethyl acetate, hexamethylphosphoramide, pyridine, sulfolane, tetrahydrofuran, or any combination thereof.

In some embodiments, the solvent (e.g., the DMSO) is added at a concentration of about 15% (v/v) to about 40% (v/v) (e.g., about 15% to 20%, about 15% to 25%, about 15% to 30%, about 15% to 35%, about 15% to 40%, about 20% to 25%, about 20% to 30%, about 20% to 35%, about 20% to 40%, about 25% to 30%, about 25% to 35%, about 25% to 40%, about 30% to 35%, about 30% to 40%). In some embodiments, solvent is added at a concentration of about 20% (v/v).

In some embodiments, the one or more AIs are non-covalently conjugated to the tobamovirus (e.g., TMGMV and/or TMV). In some embodiments, the tobamovirus (e.g., TMGMV and/or TMV) comprises one or more coat proteins that reversibly and partially dissociate in response to the presence of the solvent (e.g., DMSO). This method is further described in Example 5.

In some embodiments, the solvent is added dropwise. In some embodiments, the one or more AIs are added dropwise. In some embodiments, the one or more AIs are added stepwise over a period of time. In some embodiments, the one or more AIs are added stepwise over about 0.5 hours to about 10 days (e.g., about 0.5 to 1 hour, 0.5 to 2 hours, 0.5 to 3 hours, 0.5 to 4 hours, 0.5 to 5 hours, 0.5 to 6 hours, 0.5 to 7 hours, 0.5 to 8 hours, 0.5 to 9 hours, 0.5 to 10 hours, 0.5 to 11 hours, 0.5 to 12 hours, 0.5 to 13 hours, 0.5 to 14 hours, 0.5 to 15 hours, 0.5 to 16 hours, 0.5 to 17 hours, 0.5 to 18 hours, 0.5 to 19 hours, 0.5 to 20 hours, 0.5 to 21 hours, 0.5 to 22 hours, 0.5 to 23 hours, 0.5 to 24 hours, 0.5 hours to 2 days, 0.5 hours to 3 days, 0.5 hours to 4 days, 0.5 hours to 5 days, 0.5 hours to 6 days, 0.5 hours to 7 days, 0.5 hours to 8 days, 0.5 hours to 9 days, 0.5 hours to 9.9 days, 6 hours to 12 hours, 6 hours to 1 day, 6 hours to 2 days, 6 hours to 3 days, 6 hours to 4 days, 6 hours to 5 days, 6 hours to 6 days, 6 hours to 7 days, 6 hours to 8 days, 6 hours to 9 days, 6 hours to 10 days, 12 hours to 1 day, 12 hours to 2 days, 12 hours to 3 days, 12 hours to 12 days, 12 hours to 12 days, 12 hours to 6 days, 12 hours to 7 days, 12 hours to 8 days, 12 hours to 9 days, 12 hours to 10 days, 1 day to 2 days, 1 to 3 days, 1 to 4 days, 1 to 5 days, 1 to 6 days, 1 to 7 days, 1 to 8 days, 1 to 9 days, 1 to 10 days, 2 to 5 days, 2 to 10 days, or 5 to 10 days. In some embodiments, the one or more AIs are added once a day.

In some embodiments, the method further comprises, after adding the solvent (e.g., DMSO) and adding the one or more AIs to the tobamovirus-buffer, incubating the one or more AIs in the tobamovirus-buffer for about 0.5 hours to about 36 hours (e.g., about 0.5 to 1 hour, 0.5 to 2 hours, 0.5 to 3 hours, 0.5 to 4 hours, 0.5 to 5 hours, 0.5 to 6 hours, 0.5 to 7 hours, 0.5 to 8 hours, 0.5 to 9 hours, 0.5 to 10 hours, 0.5 to 11 hours, 0.5 to 12 hours, 0.5 to 13 hours, 0.5 to 14 hours, 0.5 to 15 hours, 0.5 to 16 hours, 0.5 to 17 hours, 0.5 to 18 hours, 0.5 to 19 hours, 0.5 to 20 hours, 0.5 to 21 hours, 0.5 to 22 hours, 0.5 to 23 hours, 0.5 to 24 hours, 0.5 to 30 hours, 0.5 to 35.9 hours, 1 to 2 hours, 1 to 3 hours, 1 to 4 hours, 1 to 5 hours, 1 to 6 hours, 1 to 7 hours, 1 to 8 hours, 1 to 9 hours, 1 to 10 hours, 1 to 11 hours, 1 to 12 hours, 1 to 13 hours, 1 to 14 hours, 1 to 15 hours, 1 to 16 hours, 1 to 17 hours, 1 to 18 hours, 1 to 19 hours, 1 to 20 hours, 1 to 21 hours, 1 to 22 hours, 1 to 23 hours, 1 to 24 hours, 1 to 30 hours, 1 to 36 hours, 2 to 3 hours, 2 to 4 hours, 2 to 5 hours, 2 to 6 hours, 2 to 7 hours, 2 to 8 hours, 2 to 9 hours, 2 to 10 hours, 2 to 11 hours, 2 to 12 hours, 2 to 13 hours, 2 to 14 hours, 2 to 15 hours, 2 to 16 hours, 2 to 17 hours, 2 to 18 hours, 2 to 19 hours, 2 to 20 hours, 2 to 22 hours, 2 to 22 hours, 2 to 23 hours, 2 to 24 hours, 2 to 30 hours, 2 to 36 hours, 3 to 4 hours, 3 to 5 hours, 3 to 6 hours, 3 to 7 hours, 3 to 8 hours, 3 to 9 hours, 3 to 10 hours, 3 to 11 hours, 3 to 12 hours, 3 to 13 hours, 3 to 14 hours, 3 to 15 hours, 3 to 16 hours, 3 to 17 hours, 3 to 18 hours, 3 to 19 hours, 3 to 20 hours, 3 to 21 hours, 3 to 22 hours, 3 to 23 hours, 3 to 24 hours, 3 to 30 hours, 3 to 36 hours, 4 to 5 hours, 4 to 6 hours, 4 to 7 hours, 4 to 8 hours, 4 to 9 hours, 4 to 10 hours, 4 to 11 hours, 4 to 12 hours, 4 to 13 hours, 4 to 14 hours, 4 to 15 hours, 4 to 16 hours, 4 to 17 hours, 4 to 18 hours, 4 to 19 hours, 4 to 20 hours, 4 to 21 hours, 4 to 22 hours, 4 to 23 hours, 4 to 24 hours, 4 to 30 hours, 4 to 36 hours, 8 to 9 hours, 8 to 10 hours, 8 to 11 hours, 8 to 12 hours, 8 to 13 hours, 8 to 18 hours, 8 to 15 hours, 8 to 16 hours, 8 to 17 hours, 8 to 18 hours, 8 to 19 hours, 8 to 20 hours, 8 to 21 hours, 8 to 22 hours, 8 to 23 hours, 8 to 24 hours, 8 to 30 hours, 8 to 36 hours, 12 to 13 hours, 12 to 18 hours, 12 to 15 hours, 12 to 16 hours, 12 to 17 hours, 12 to 18 hours, 12 to 19 hours, 12 to 20 hours, 12 to 21 hours, 12 to 22 hours, 12 to 23 hours, 12 to 24 hours, 12 to 30 hours, 12 to 36 hours, 24 to 30 hours, or 12 to 36 hours).

In some embodiments, after adding the solvent and the one or more AIs, the solution in which the nanoparticles are purified in has a pH of about 5 to about 9 (e.g., about 5 to 6, 5 to 7, 5 to 8, 5 to 8.9, 6 to 7, 6 to 8, 6 to 9, 7 to 8, 7 to 9, 8 to 9, 5.5 to 6.5, 5.5 to 7.5, 5.5 to 8.5, 5.5 to 8.9, 6.5 to 7.5, 6.5 to 8.5, 6.5 to 8.9, 7.5 to 8.5, 7.5 to 9, 8.5 to 9, 6.9 to 7.1, 6.9 to 7.2, 6.9 to 7.3, 5 to 6.5, 5 to 7.5, 5 to 8.5, 5 to 8.9, 6 to 7.5, 6 to 8.5, 6 to 9.5, 7 to 7.1, 7 to 7.2, 7 to 7.3, 7 to 7.4, 7 to 7.5, 7 to 7.6, 7 to 7.7, 7 to 7.8, 7 to 7.9, 7 to 9.5, 8 to 9.5, 5.5 to 6, 5.5 to 7, 5.5 to 8, 5.5 to 8.9, 6.5 to 7, 6.5 to 8, 6.5 to 9, or 7.5 to 8). In some embodiments, the solution in which the nanoparticles are purified in has a pH of about 6.9, 7.0, 7.1, 7.2, or 7.3.

In some embodiments, the one or more AIs are added to the tobamovirus-buffer two or more times when preparing the nanoparticles using the pH method or the solvent method. In some embodiments, the one or more AIs are added at least once a day. In some embodiments, the one or more AIs are added dropwise, only once a day when preparing the nanoparticles using the pH method or the solvent method. In some embodiments, when preparing the nanoparticles using the pH method or the solvent method, the one or more AIs are added until reaching an equivalence ratio of about 10:1, 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, or 1000:1. In some embodiments, when preparing the nanoparticles using the pH method or the solvent method, the one or more AIs are added in 1,000, 1,500, 2,000, 2,500, 3,000, 3,3,00, 4,000, 4,500, 5,000, 5,500, 6,500, 7,000, 7,500, 8,000, 8,500, 9,000, or 9,500-fold molar excess to the tobamovirus (e.g., TMGMV and/or TMV). In some embodiments, when preparing the nanoparticles using the pH method or the solvent method, 100, 150, 200, 250, 300, 350, 400, 450, or 500 nmol of one or more AIs per gram of tobamovirus (e.g., TMGMV and/or TMV) is added.

Methods for formulating and delivering a composition to soil, crops, and plants are well known in the art (U.S. Pat. Nos. 5,091,188; 5,091,187; 5,250,236; 5,472,706; 5,750,142; 5,874,029; 5,879,715; 4,725,442; 6,835,396; 6,872,773; 8,404,263; 9,095,133; PCT applications WO 2005/102507; WO 2005/020933; WO 2005/072680; WO 01/88046; WO 2007/014826; US application 2005/0170004; 2006/0063676; 2014/164418). The disclosed formulations may be mixed in any order in a single or multistep mixing. One or more of compounds/AIs may be added to the formulation comprising a tobamovirus (e.g., TMGMV and/or TMV) nanoparticle and examples of suitable agrochemical formulation are liquid formulations such as EC (Emulsifiable concentrate) formulation; SL or LS (Soluble concentrate) formulation; EW (Emulsion, oil in water) formulation; ME (Microemulsion) formulation; MEC (Microemulsifiable concentrates) formulation; CS (Capsule suspension) formulation; TK (Technical concentrate) formulation; OD (oil based suspension concentrate) formulation; SC (suspension concentrate) formulation; SE (Suspo-emulsion) formulation; ULV (Ultra-low volume liquid) formulation; SO (Spreading oil) formulation; AL (Any other liquid) formulation; LA (Lacquer) formulation; DC (Dispersible concentrate) formulation; or solid formulations such as WG (Water dispersible granules) formulation; TB (Tablet) formulation; FG (Fine granule) formulation; MG (Microgranule) formulation; SG (soluble Granule). In some embodiments, liquid formulations are EC, SL, LS, EW, ME, MEC, TK, OD, SC, SE, ULV, SO, AL, LA and DC.

### Pharmaceutical Compositions

Disclosed herein, in certain embodiments, are pharmaceutical compositions comprising a nanoparticles as described herein. Two or more (e.g., two, three, or four) of any of the types of therapeutic nanoparticles described herein can be present in a pharmaceutical composition in any combination. The pharmaceutical compositions can be formulated in any manner known in the art.

In some embodiments, the pharmaceutical composition comprises at least one pharmaceutically acceptable carrier, diluent, or excipient. In some embodiments, the pharmaceutical composition is formulated into a dosage form that is an injectable solution, a lyophilized powder, a suspension, or any combination thereof.

Pharmaceutical compositions are formulated to be compatible with their intended route of administration (e.g., intravenous, intraarterial, intramuscular, intradermal, subcutaneous, or intraperitoneal). In some embodiments, the compositions provided herein can include a pharmaceutically acceptable diluent (e.g., a sterile diluent). In some embodiments, the pharmaceutically acceptable diluent can be sterile water, sterile saline, a fixed oil, polyethylene glycol, glycerine, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfite, chelating agents such as ethylenediaminetetraacetic acid, buffers such as acetates, citrates, or phosphates, and isotonic agents such as sugars (e.g., dextrose), polyalcohols (e.g., mannitol or sorbitol), or salts (e.g., sodium chloride), or any combination thereof.

In some embodiments, the pharmaceutical compositions provided herein can include a pharmaceutically acceptable carrier. Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the nanoparticles can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing one or more of any of the nanoparticles described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage). In some embodiments, the compositions containing one or more of any of the nanoparticles described herein can be formulated into a dosage form that is an injectable, a lyophilized powder, a suspension, or any combination thereof.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) nanoparticles (e.g., any of the nanoparticles described herein) can be an amount that decreases cancer cell invasion or metastasis in a subject having cancer in a subject (e.g., a human), or decreases and/or eliminates an infection in a subject (e.g., a human).

The effectiveness and dosing of any of the nanoparticles described herein can be determined by a health care professional using methods known in the art, as well as by the observation of one or more symptoms of the disease (e.g., cancer or an infection) in a subject (e.g., a human). Certain factors may influence the dosage and timing required to effectively treat a subject (e.g., the severity of the disease or disorder, previous treatments, the general health and/or age of the subject, and the presence of other diseases).

One of ordinary skill in the art will understand that therapeutic agents, including the nanoparticles described herein, vary in their potency, and effective amounts can be determined by methods known in the art. Typically, relatively low doses are administered at first, and the attending health care professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the nanoparticles in vivo.

The pharmaceutical compositions can be included in a kit, container, pack, or dispenser together with instructions for administration.

### Methods of Treatment

Also provided herein are methods of treating cancer in a subject in need thereof. The method of treating cancer comprises administering a nanoparticle of the disclosure or a pharmaceutical composition of the disclosure to the subject in need of treatment for cancer. In some embodiments, the nanoparticle or the pharmaceutical composition is administered in an effective amount. In some embodiments, the cancer comprises breast cancer, ovarian cancer, glioma, gastrointestinal cancer, prostate cancer, carcinoma, lung carcinoma, hepatocellular carcinoma, testicular cancer, cervical cancer, endometrial cancer, bladder cancer, head and neck cancer, lung cancer, gastro-esophageal cancer, gynecological cancer, or any combination thereof.

Also provided herein are methods of treating an infection in a subject in need thereof, the method of treating an infection comprises administering a nanoparticle of the disclosure or a pharmaceutical composition of the disclosure to the subject in need of treatment for the infection. In some embodiments, the nanoparticle or the pharmaceutical composition is administered in an effective amount. In some embodiments, the infection is a bacterial infection, a viral infection, a fungal infection, a parasitic infection, or any combination thereof.

### EXAMPLES

The disclosure is further described in the following examples, which do not limit the scope of any embodiments described in the claims.

### Methods

### Preparation of TMGMV

TMGMV was obtained from BioProdex (Gainesville, FL, USA) and stored at −20° C. until use. The solution was thawed at 4° C. overnight and then dialyzed against potassium phosphate buffer (KP; 10 mM, pH 7.2) for 24 hours at 4° C. using 12-14 kDa dialysis tubing (Fisher Scientific S432700; Waltham, MA, USA). The buffer solution was replaced, and the dialysis continued for an additional 48 hours. The solution was then centrifuged at 10,000×g for 20 min (Beckman Coulter Allegra or Avanti centrifuges). The supernatant was collected and ultracentrifuged at 42,000 rpm for 2.5 hours at 4° C. (Beckman Coulter Optima L-90k Ultracentrifuge with 50.2 Ti rotor; Brea, CA, USA). The pellet was resuspended under rotational mixing overnight at 4° C. in KP buffer. The sample concentration was then confirmed using a Nanodrop 2000 (Thermo Scientific; Waltham, MA, USA); the concentration was adjusted to 10 mg mL⁻¹ in 10 mM KP before storing at 4° C. (for TMGMV CP, 6260=3 mL mg⁻¹ cm⁻¹)

Preparation of Diazonium Salt from 4-Ethynylaniline

In a 5 mL tube, 298 mg of 4-ethynylaniline was dissolved in 2 mL methanol. In a 50 mL tube, 1.09 g p-toluenesulfonic acid was dissolved in 20 mL DIH₂O. Both solutions were placed at −20° C. for 10 minutes to precool. A solution of 1.5 mL of 3M sodium nitrite (258 mg in 1.5 mL DIH₂O) was prepared and placed at −20° C. for 5 minutes to precool. A 50 mL beaker was submerged in ice/water slurry on a stir plate. The solutions were removed from the freezer. A stir bar was added to the 20 mL of precooled acid in the submerged beaker. Once mixing, the methanol solution was added. The solution turned opaque and beige in color. The nitrite solution was gradually dropped into the acid solution and the mixture gradually turned yellow and eventually turned red after 30-60 minutes of reaction time. A sample of 1 mL of the diazonium slurry was collected and centrifuged for 2 minutes at 10,000×g to isolate diazonium salts. On ice, the supernatant was removed and the diazonium salts were resuspended in 1 mL of precooled ethanol. The prepared diazonium salts were used immediately for tyrosine modification.

### Coupling of Diazonium to TMGMV

A solution of 962 μL of 2 mg mL⁻¹ TMGMV in 100 mM borate buffer (pH 8.5) was prepared and precooled on ice. The diazonium salt solution was added to the TMGMV solution at a volume of 80 μL. The solution was mixed by inversion and reacted on ice for 30 minutes. The solution was centrifuged at 50,000 rpm in the tabletop ultracentrifuge (Beckman Optima MAX-XP with TLA-55 rotor) for 1 hour on a sucrose cushion (30% w/v). The viral pellet was resuspended in 10 mM KP overnight at 4° C. on a rotary shaker.

### Copper-Catalyzed Azide-Alkyne Cycloaddition Reaction

To an ultracentrifugation tube (Beckman Coulter 357448, Indianapolis, IN, USA), 1 mg of TMGMV was added. The reaction medium consisted of 1 mM copper sulfate, 2 mM aminoguanidine, 2 mM L-ascorbic acid, and 3.7 mM tris(benzyltriazolylmethyl)amine. Fifty equivalences of 6A-Azido-6A-deoxy-b-cyclodextrin (TCI Chemicals) per TMGMV coat protein were added, and the volume of 10 mM KP pH 7 was adjusted for a final volume of 500 μL. The reaction was left to progress for 1 hour on ice. To the bottom of the same tube, a 200 μL sucrose cushion (30% w/v) was added, and the sample was then ultracentrifuged at 50,000 rpm for 1 hour at 4° C. (Beckman Optima MAX-XP with TLA-55 rotor). The supernatant was removed and the pellet was resuspended under rotational mixing at 4° C. overnight before further characterization.

### Characterization of Chemically Labeled β-CD-TMGMV

SDS-PAGE: Denatured β-CD-conjugated TMGMV samples (10 μg) were loaded on a 12% NuPAGE gel (Life Technologies) and run on 1×MOPS Running Buffer (Life Technologies). Gel Code Blue stain (Life Technologies) was used to stain proteins and visualized under white light.

FPLC (Size exclusion chromatography): β-CD-conjugated TMGMV samples (500 μL at 0.5 mg/mL) were analyzed using a Superose6 Increase 100 GL column and an ÄKTA Pure25 chromatography system (GE Healthcare) using a flow rate 0.5 mL/min in 10 mM KP (pH 7.4). The absorbance at 260 and 280 nm was recorded.

TEM imaging: Samples were diluted to the concentration of 0.05 mg mL⁻¹ and absorbed onto carbon-coated TEM grids (Electron Microscopy Sciences). The grids were then washed three times with pure water. Then, grids were stained by 2% (w/v) uranyl acetate for 2 min for imaging. TEM was conducted using a FEI Tecnai F30 transmission electron microscope operated at 300 kV.

### Loading of Pesticides to β-CD-TMGMV

To assess loading of pesticides to β-CD-TMGMV, competition assays were completed between Doxorubicin (ApexBio) and the target molecules. Samples of 100 μL volume were prepared in a 96-well plate (Costar). For sample wells, 0.0825 mg of β-CD-conjugated TMGMV in KP buffer were added to the wells, in addition with doxorubicin at 10 eq_{β-CD-TMIGMV} (molar equivalents to β-CD-conjugated TMGMV).

Three control conditions were utilized: 1 eq TMGMV, 575 eq β-CD, and 1 eq TMGMV with 575 eq β-CD (non-conjugated). These control wells also received doxorubicin at 10 eq. The plate was incubated overnight at 4° C. on a plate rocker (Fisher). Fluorescence top readings were conducted on the plate (excitation 470 nm, emission 595 nm, 25 flashes) via UV-Vis Plate Reader (Tecan infinite 200Pro). Upon completion of fluorescence measurements, β-CD-TMGMV and control wells received either Clothianidin (BASF), Fluopyram (BASF), or Tetracycline (Sigma-Aldrich) at 0, 10, 100, or 1000 eq_{β-CD-TMGMV}. Following a repeat overnight incubation at 4° C. on a plate rocker (Fisher), fluorescent reading was repeated as before.

Samples of 100 μL volume were prepared in a 96-well plate (Costar). For sample wells, 0.0825 mg of β-CD-conjugated TMGMV in KP buffer were added to the wells, in addition with doxorubicin at 10 eq (molar equivalents to β-CD-conjugated TMGMV). Three control conditions were utilized: 1 eq TMGMV, 575 eq β-CD, and 1 eq TMGMV with 575 eq β-CD (non-conjugated). These control wells also received doxorubicin at 10 eq. Overnight incubation at 4° C. on a plate rocker (Fisher) allowed for loading of doxorubicin onto β-CD-TMGMV particles. Fluorescence top readings were then conducted on the plate (excitation 470 nm, emission 595 nm, 25 flashes) via UV-Vis Plate Reader (Tecan infinite 200Pro). Upon completion of fluorescence measurements, β-CD-TMGMV and control wells received either Clothianidin (CTD; BASF), Fluopyram (FLP; BASF), or Tetracycline (TET; Sigma-Aldrich) at 10, 100, or 1000 eq. Repeat overnight incubation at 4° C. then allowed for these molecules to compete against doxorubicin for entrapment on the β-CD-TMGMV. The fluorescence was then read on the plate reader as described before.

### Partial Dissociation (Also Called “Breathing”)

TMGMV at a concentration of 5 mg mL⁻¹ in KP buffer at pH 7.5 was kept for 5 days at 4° C. Addition of the target AI to the solution was done every 24 h until reaching a 500:1 equivalence ratio and left mixing on a rotary shaker. Afterwards, the solution was centrifuged at 50,000 rpm in the tabletop ultracentrifuge (Beckman Optima MAX-XP with TLA-55 rotor) for 1 hour on a sucrose cushion (30% w/v). The viral pellet was then resuspended in 10 mM KP pH 7 overnight at 4° C. on a rotary shaker. After total resuspension, the solution was dialyzed for 48 h to remove excess (non-entrapped) AI.

### TEM Imaging

Samples were diluted to the concentration of 0.05 mg mL⁻¹ and absorbed onto carbon-coated TEM grids (Electron Microscopy Sciences). The grids were then washed three times with pure water. Then, grids were stained by 2% (w/v) uranyl acetate for 2 min for imaging. TEM was conducted using a FEI Tecnai F30 transmission electron microscope operated at 300 kV.

### Soil Mobility Assays

Garden Magic Top Soil was packed at a density of 0.32 g cm⁻³ into a cylindrical column (28 mm diameter, top height 30 cm) and saturated with deionized water to remove air pockets. The density of soil in real environments can be higher (0.6-1.6 g cm-3) due to compaction effects with depth and over time. A bolus that contained 1 mg of each formulation with and without conjugated or infused dye molecules was injected at the top of the soil column and saturated the column with deionized water at a constant flow rate of 1.5 cm³ min⁻¹. The eluent was collected at the base of the column in 500 μl-2 mL fractions. Up to 200 fractions were collected in each trial (two trials per depth for each formulation).

The elution fractions were analyzed by SDS-PAGE to determine the mass and amount of nanoparticles recovered in each elution fraction. TMGMV nanoparticles were analyzed on 4-12% NuPage polyacrylamide SDS gels cast according to the Surecast Handcast protocol (Invitrogen). 25 μl of each elution fraction were mixed with 5 μl 5×SDS loading buffer and separated the samples for 1 h at 200 V and 120 mA with SeeBlue Plus2 ladder size. The gels were then stained with Gel Code Blue Stain (Life Technologies) and microwaved for 1 min and then agitated for 5 minutes. Then, the process was repeated with deionized water for de-staining. The gels were imaged using the FluorChem R system.

All the nanoparticles were imaged and analyzed using ImageJ.

Infusion of Hydrophobic Cargo in TMGMV by pH Increase from pH 7 to pH 7.5

Loading of AI into TMGMV via the “pH method” was performed. TMGMV at a concentration of 1 mg mL⁻¹ in 10 mM KP buffer at pH 7.5 was kept for 10 days at 4° C. The following AIs were used: fluopyram and clothianidin, (BASF, Berkeley, CA, USA), rifampicin and ivermectin (BioVision; Milpitas, CA, USA). Cy5 (Lumiprobe; Cockeysville, MD, USA) and doxorubicin (ApexBio; Houston, TX, USA) were also studied as proof of concept (fluorescent molecule and cancer chemotherapy). The AI was added to TMGMV by adding an excess of 10:1 AI:coat protein (CP; each TMGMV rod is assembled from ˜2,100 identical CPs) every day until a ratio of 100:1 was reached. During this process the reaction was kept mixing on a rotary shaker. 1 mL aliquots were obtained each day for further analysis.

After AI loading, the aliquots were spin-filtered using 100K molecular weight cut-off 0.5 mL filters (MilliporeSigma, Burlington, MA, USA). 200 μL of the aliquot and 250 μL of KP solution were added and then centrifuged at 16,160×g for 5 minutes at 4° C., the flow-through was discarded, and then 450 μL of KP was added and centrifuged again, this step was repeated 3 times. After the third centrifugation, the filter was inverted in a new tube and centrifuged at 1000×g for 2 minutes, to recover the supernatant and carry out the subsequent characterization.

### Infusion of Hydrophobic Cargo in TMGMV by Change in DMSO Concentration

Loading of AI into TMGMV via the “DMSO method” was performed. TMGMV in 10 mM KP buffer (pH 7.2) was diluted to 5 mg mL-1 in 2 mL of buffer and transferred to a 25 mL beaker and magnetically stirred at 300 rpm at room temperature. A solution of DMSO and 10 mM KP was added dropwise to dilute the solution to a 20% (v/v) concentration of DMSO and 2 mg mL-1 of TMGMV. Aliquots of the AIs were added dropwise to the solution to prevent precipitation. The solutions were left to stir at room temperature for 24 hours. The samples were collected and spin filtered as described above before being stored at 4° C.

### TEM

TMGMV samples were diluted to a concentration of 0.05 mg mL-1 and absorbed onto carbon-coated TEM grids (Electron Microscopy Sciences, Hatfield, PA, USA). The grids were then washed three times with pure water. Then, grids were stained by 2% (w/v) uranyl acetate for 90 seconds. TEM was conducted using a FEI Tecnai F30 transmission electron microscope operated at 300 kV. Image analysis was performed using ImageJ software (https://imagej.nih.gov/ij/download.html). To determine the change in width of the nanoparticles, the width of sections of 100 nm in length was measured for standardization purposes. Five different sections were measured per micrography and 30 of them in total per sample. Subsequently, for the complete particle, the length, the perimeter and the area were measured, to later calculate the average width from the perimeter.

### Size Exclusion Chromatography

TMGMV samples (500 μL at 0.5 mg/mL) were analyzed using a Superose6 Increase 100 GL column and an AKTA Pure25 chromatography system (GE Healthcare, Chicago, Il, USA) using a flow rate 0.5 mL/min in 10 mM KP (pH 7.4). The absorbance at 260 and 280 nm was recorded.

### Circular Dichroism Spectroscopy

CD spectra were obtained using an Aviv model 215 CD spectrometer (Lakewood, NJ, USA). All samples were run in a quartz cuvette with a path length of 2 mm (Stama Cells, Atascadero, CA, USA) at 25° C. Samples were dissolved in a 10 mM KP buffer at pH 7 to a concentration ranging from 0.025 mg/mL to 0.5 mg/mL to obtain a volume of 400 μL for each CD run. Near and far UV spectra were obtained in separate scans. For far UV spectra, samples were scanned from 250 nm to 180 nm with a wavelength step of 1 nm and an averaging time of 1 second. For near UV spectra, samples were scanned from 310 nm to 240 nm with a wavelength step size of 0.5 nm and an averaging time of 1 second. All spectra were scanned twice and averaged within each UV region.

### Small Molecules Quantification by High-Performance Liquid Chromatography (HPLC)

For HPLC, AI was extracted from TMGMV. In brief, the concentration of TMGMV samples was adjusted to 1.2 mg mL-1 in KP. The solution was diluted 4-fold into a 1:1 acetonitrile/:methanol mixture and vortexed for 30 seconds. The solution was centrifuged at 10,000×g for 10 minutes at 4° C. and the organic phase (bottom fraction) was collected and transferred to an HPLC 2 mL glass screw top vial (SureSTART, Thermo Scientific, Waltham, MA, USA).

After a 10-fold dilution in acetonitrile, the extracted samples were injected at 500 μL and run on 5 μm C18 column (20×100 mm) using a Shimadzu LC-40 HPLC system (Columbia, MD, USA). The method was run at 0.5 mL min-1 in a gradient of acetonitrile and 0.02% (v/v) phosphoric acid for 15 minutes per sample. A photodiode array was used to collect absorbance values at 280 nm (fluopyram), 269 nm (clothianidin), 225 nm (ivermectin), and 330 nm (rifampicin). The absorbance values were fitted to a standard curve to identify sample concentration with N=3.

### Example 1: β-CD-TMGMV Formation

Experiments were performed to test the formation and integrity of the conjugated TMGMV particles before AI loading.

SDS-PAGE confirmed the covalent attachment of β-CD to the CP as indicated by the additional higher molecular weight (MW) band at lane 3 in FIG. 2; the MW increase corresponds to the size of β-CD. The conjugation efficiency was roughly estimated to be about 35% based on band density analysis by ImageJ of β-CD-TMGMV subunit proteins versus TMGMV and TMGMV-Alkyne. This suggests that when using a molar excess of 50:1 of β-CD-to-CP, conjugation yielded approximately 750 molecules per particle.

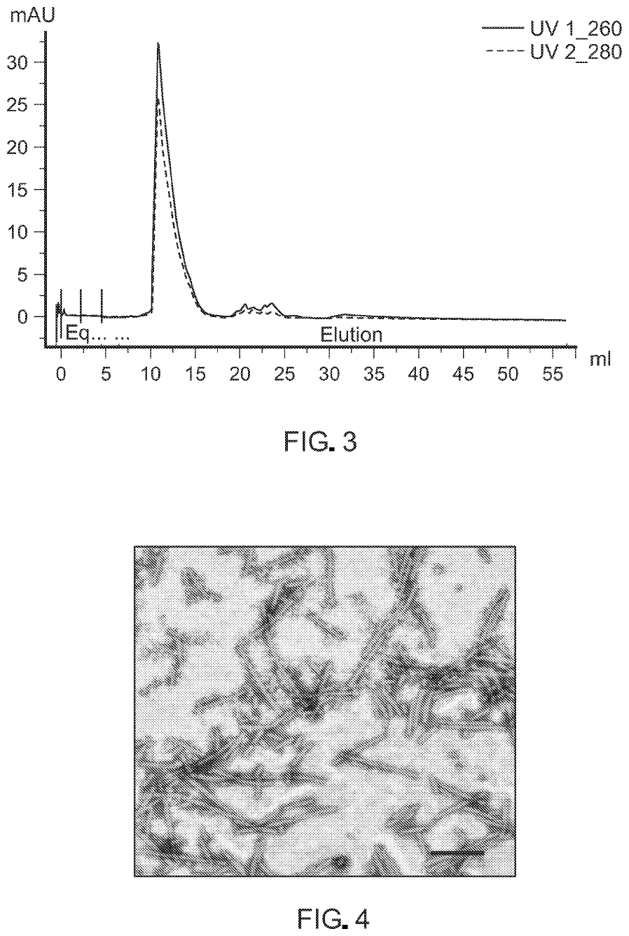

To verify structural integrity of the modified TMGMV particles, size exclusion chromatography (SEC) and transmission electron microscopy (TEM) were performed. As seen in FIG. 3, SEC measurements showed no significant difference between native and conjugated TMGMV (shown below), showing an elution volume around 9 mL, as the native one and an A260:280 ratio of 1.2, indicative of intact TMGMV. Furthermore, no obvious signs of aggregation or particle dissociation were observed. As seen in FIG. 4, TEM imaging confirmed the structural integrity of 0-CD-TMGMV after modification and purification.

### Example 2: AI Loading to β-CD-TMGMV

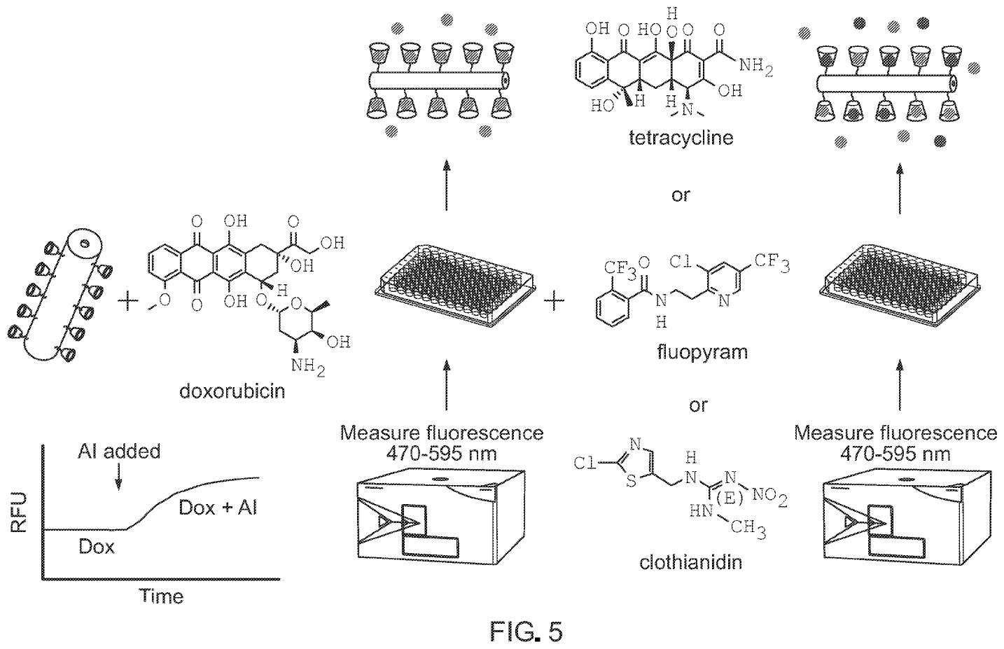

Experiments were conducted to test AI loading (or “entrapment”) to β-CD-TMGMV. Quantifying the loading (“entrapment”) of pesticides presented numerous difficulties due to the molecules' lack of fluorescence. To quantify the entrapment, a competition assay was performed (see schematic depiction of FIG. 5). Doxorubicin (“DOX”), with a known excitation (470 nm) and emission (595 nm) wavelengths, was added to the sample and control wells. While free doxorubicin is able to fluoresce at 595 nm, entrapped doxorubicin does not; this disparity allows for a measurement of the free doxorubicin in solution (and indirect quantification of the entrapped molecules).

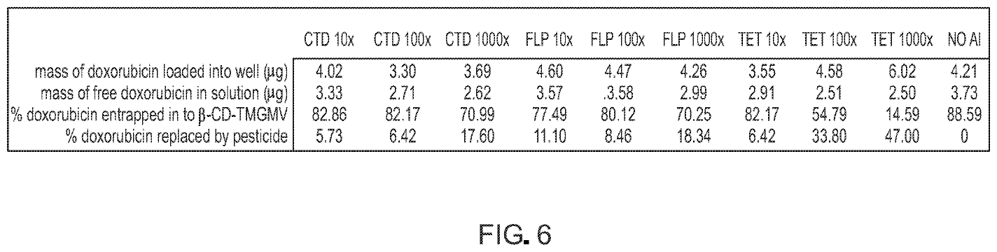

By measuring the fluorescence of the non-entrapped doxorubicin, it was determined that 88.59% of what was loaded into the well was entrapped by the β-CD-TMGMV. This entrapment, measured as a decrease in the fluorescence of doxorubicin (as it is pulled out of solution), affirms molecular carrying capacity of 3-CD-TMGMV. As expected, the addition of a pesticide resulted in a dose-dependent displacement of doxorubicin from β-CD-TMGMV, with larger concentrations of pesticide displacing more doxorubicin. This increase in the amount of free doxorubicin in the well resulted in increased relative fluorescence. When challenged by the addition of Clothianidin (“CTD”), Fluopyram (“FLP”), or Tetracycline (“TET”), as seen in the table in FIG. 6, Tetracycline at 1000 eq displaced 47% of the doxorubicin that had initially been bound to β-CD-TMGMV. Clothianidin and Fluopyram at 1000 eq displaced 17.60 and 18.34% of the doxorubicin, respectively. These data correlate with the degree of hydrophobicity of the molecules, and as expected, the β-CD acted as a bucket to entrap the hydrophobic molecules with the most ease.

### Example 3: AI Loading to TMGMV

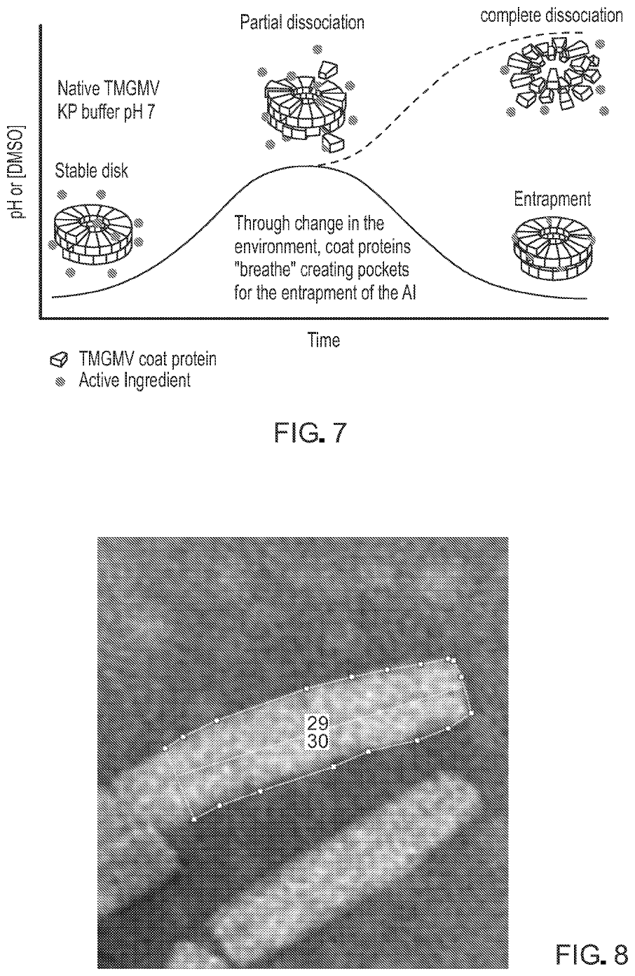

Experiments were conducted to load pesticides into TMGMV by strategically altering the pH. Without being bound by theory, this entraps Als through the formation of “pockets” between coat proteins (CPs). Without being bound by theory, the rationale is that by increasing the pH of the buffer, the virus will start to dissociate and hydrophobic pockets will get created between the virion's coat proteins. AIs are then added to interact with the virus particles and then the pH is decreased to promote particles' self-assembly and entrapment of AI on the hydrophobic pockets (see schematic of FIG. 7). As discussed above, TMGMV at a concentration of 5 mg mL-1 in KP buffer at pH 7.5 was kept for at 4° C. for 5 days, and the target AI was added to the solution every 24 h until reaching a 500:1 equivalence ratio. Afterwards, the solution was centrifuged at 50,000 rpm for 1 hour on a sucrose cushion (30% w/v). The viral pellet was then resuspended in 10 mM KP pH 7 overnight at 4° C., and after total resuspension, the solution was dialyzed for 48 h to remove excess (non-entrapped) AI. Samples were then observed at TEM and analyzed using ImageJ.

To determine the change in width (breathing/infusion) of the nanoparticles, the width of sections of 100 nm in length of nanoparticles (as shown in the micrography of FIG. 8) was measured. This was done to standardize the measurements and determine the change in width. At least 5 different sections were measured per micrography, and at least 30 sections were measured in total per sample.

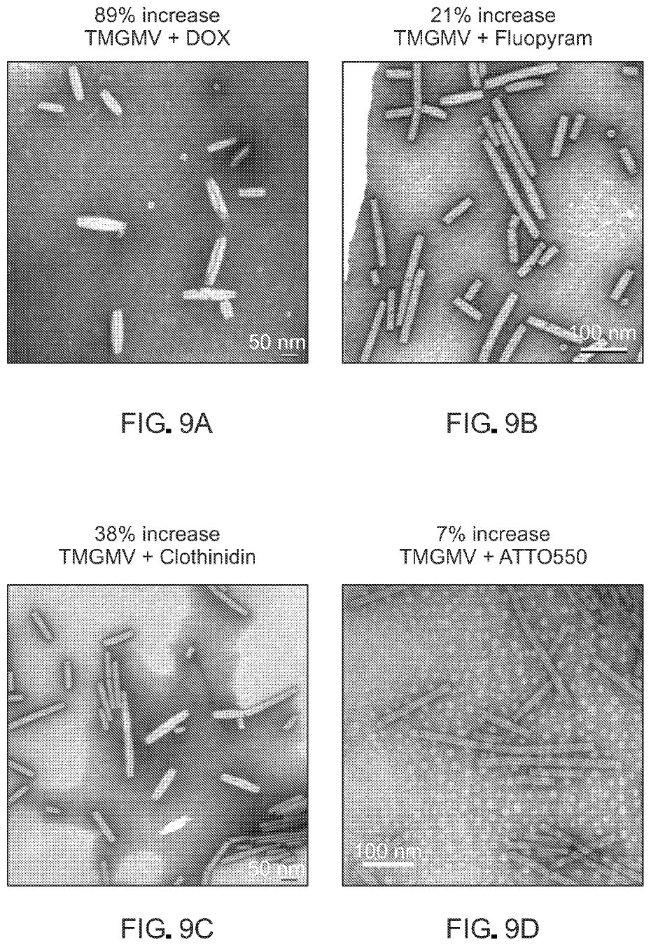

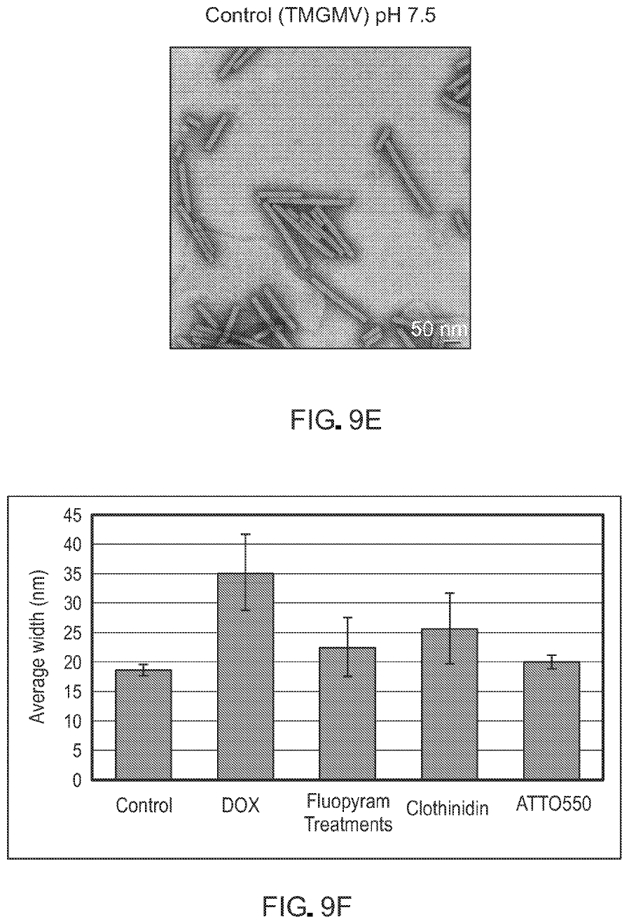

After the measurements, the differences between native TMGMV (control) vs. infused with doxorubicin, ATTO550, fluopyram, and clothianidin (FIGS. 9A-9F) were determined. The greatest increase was observed with doxorubicin, with an average width of 35 nm, compared to 18 nm of the control (DOX: 89% increase; FIG. 9A compared to control FIG. 9E). Clothianidin showed the second greatest increase of 38% (FIG. 9C; 26 nm vs. 18 nm of the control). Fluopyram had an increase of 21% in width (FIG. 9B; 22 nm vs. 18 nm), while ATTO550, the most hydrophilic of the AIs 15 showed the smallest change with a 7% increase in width (FIG. 9D). This would correlate to pockets entrapping the hydrophobic compounds better.

### Example 4: Soil Mobility of the TMGMV Nanoparticles

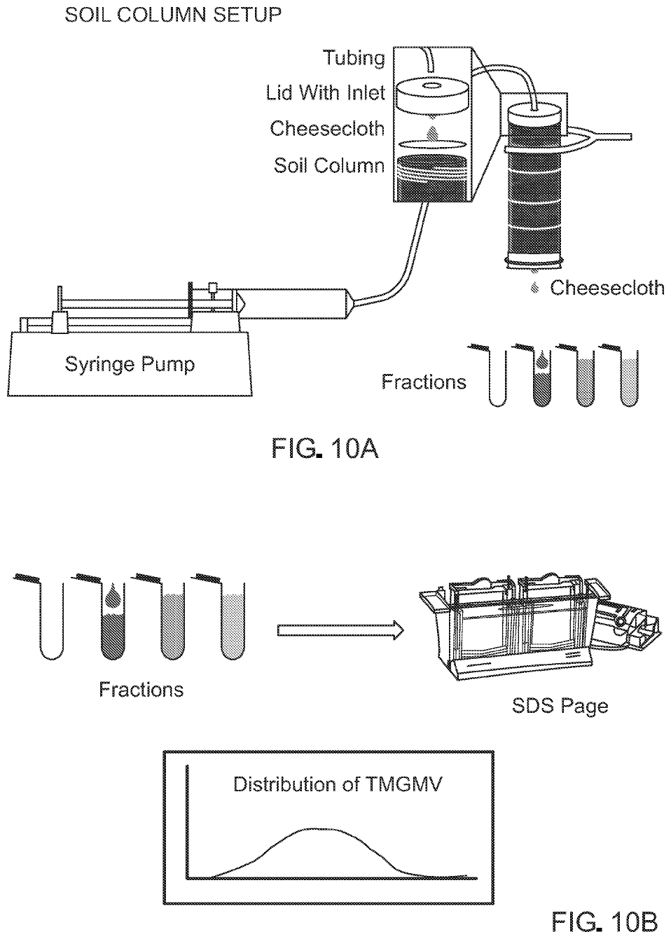

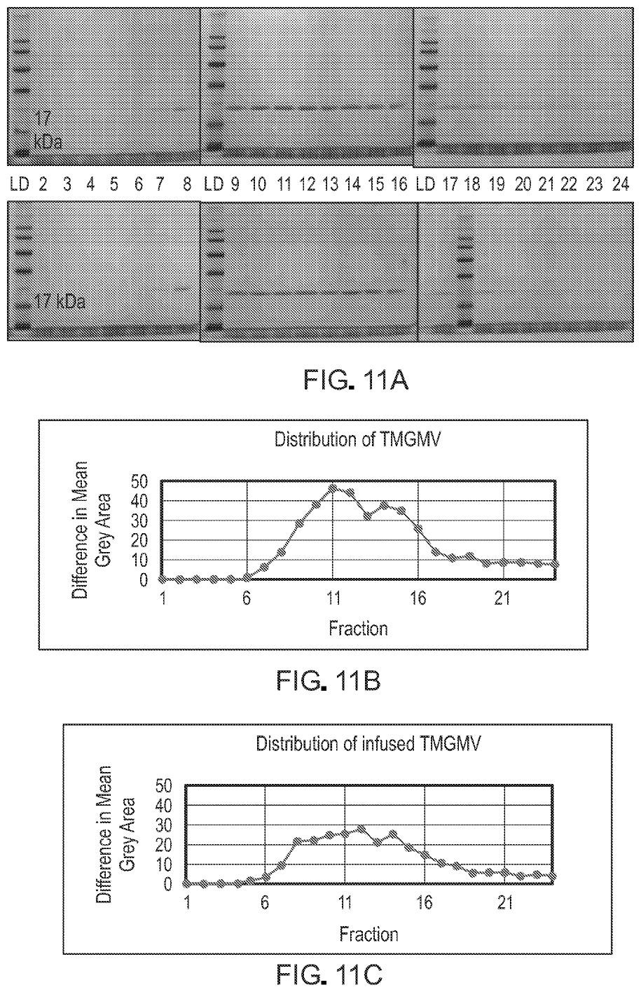

Experiments were conducted to test the soil mobility of various TMGMV nanoparticles. The experimental set-up is depicted in the schematic of FIG. 10A. The soil column set-up comprised cheesecloth, which prevented depression formation on top of the soil. The fractions were collected and analyzed via SDS-PAGE as described above (depicted in FIG. 10B). The soil was treated with TMGMV and infused TMGMV, which had undergone 5 days of treatment in a buffer with a higher pH and placed back into a buffer. The gels were imaged (FIG. 11A) and quantified, and the results showed that the infused TMGMV nanoparticle (FIG. 11C) has the same penetration ability to that of TMGMV alone (FIG. 11B). This finding confirmed that the TMGMV “breathing” technique worked and that it does not effect the mobility of the nanoparticles.

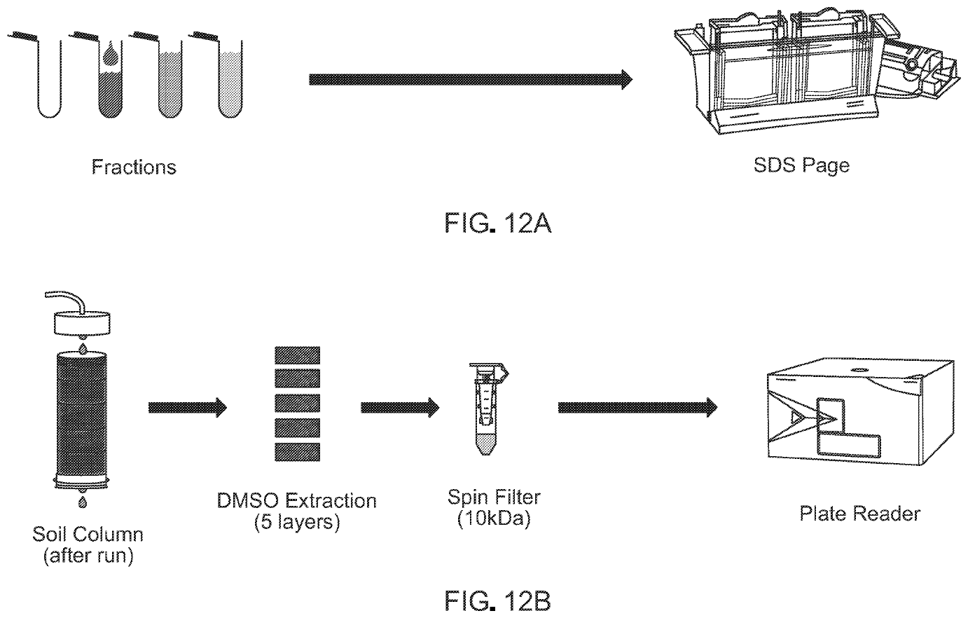

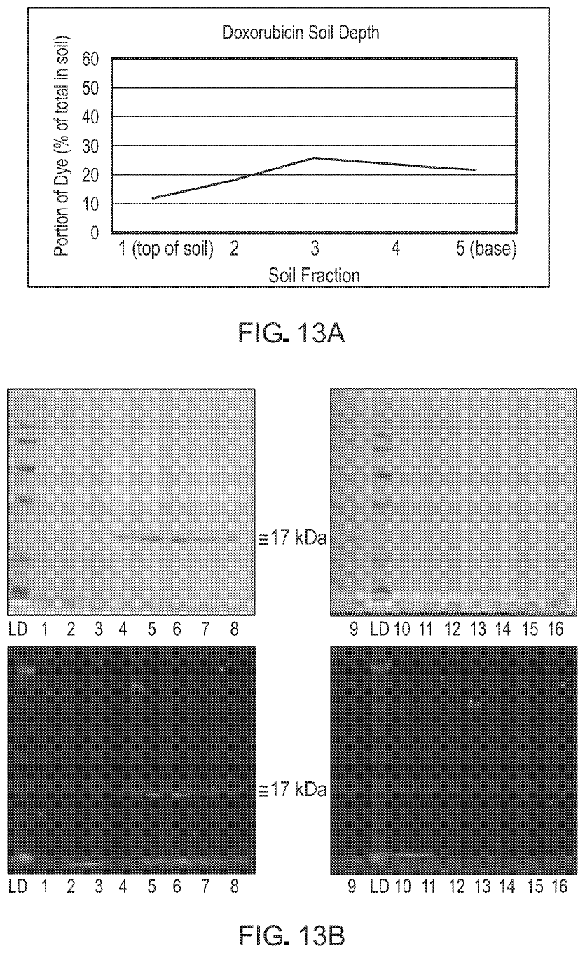

TMGMV-DOX nanoparticles were made by loading doxorubicin onto TMGMV as described above and analyzed on a soil column via SDS-PAGE and a plate reader (as depicted in FIGS. 12A-12B, respectively). The soil column is 30 cm in length and was divided it into 5 fractions, each fraction was 6 cm (depicted in FIG. 12B). Therefore, the first fraction represented the first 6 cm of soil nearest the top of the column, the 3rd fraction (middle one) would be 12-18 cm deep, and the 5th fraction would be 24-30 cm deep. The TMGMV-DOX nanoparticle was found present in all five of the fractions (FIG. 13A). The highest percentage of the TMGMV-DOX nanoparticles were found in the 3^{rd} fraction, representing the middle of the soil column, but over 20% of the nanoparticles were present in the 5^{th} fraction, the bottom of the soil column representing deep penetration, and over 10% of the TMGMV-DOX nanoparticles were found in the 1s fraction, nearest to the top of the soil. The results are supported by the gel analysis of the fractions (FIG. 13B).

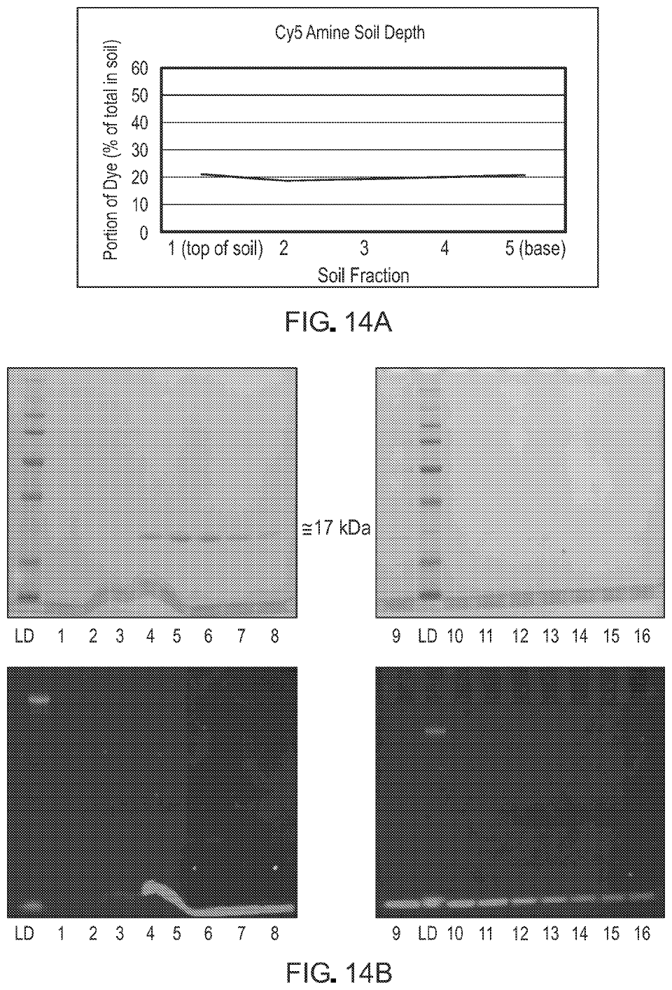

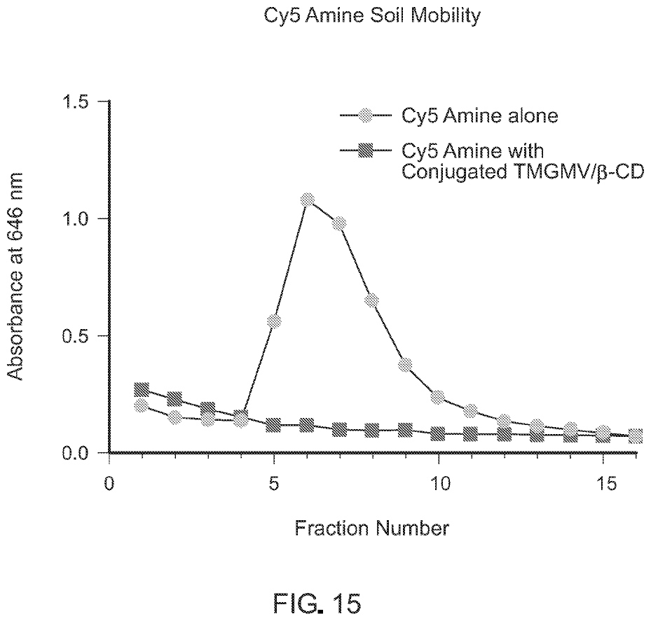

TMGMV-Cy5 nanoparticles were made by loading Cy5 amine onto TMGMV as described above and analyzed on a soil column via SDS-PAGE and a plate reader (as depicted in FIGS. 12A-12B, respectively). As described above, the column was split into five fractions, and interestingly, the TMGMV-Cy5 was evenly dispersed throughout all the fractions (FIG. 14A). About 20% of the nanoparticles were present in the all five of the fractions (FIG. 14A). The results are supported by the gel analysis of the soil fractions (FIG. 14B).

The soil mobility of TMGMV-βCD nanoparticles loaded with Cy5 was also tested and the results showed that the loaded nanoparticles were also evenly distributed throughout the soil. The results showed no elution and the nanoparticles were primarily retained by the soil. As a reference, Cy5 was run through a soil column. Cy5 was unable to penetrate much into the soil and was primarily located on top of the soil. This is supported by studies showing that Cy5 cannot penetrate further than 4 cm into soil because it bound strongly to the soil particles (Chariou, et al. (2019) Nat. Nanotechnol. 14:712). These results are comparable to data reported for abamectin (Chariou & Steinmetz (2017) ACS Nano 11, 4719), fenamiphos and oxamyl (Hassan, et al. (2016) Plant Pathol. J 15, 144), as well as other pesticides (Pestovsky & Martinez-Antonio (2017) J. Nanosci. Nanotechnol. 17, 8699).

No matter which nanoparticle type was used as a carrier, the mobility of Cy5 within the column was significantly enhanced and retained in the soil better than Cy5 alone.

### Example 5: AI Loading to TMGMV Via pH and DMSO Methods

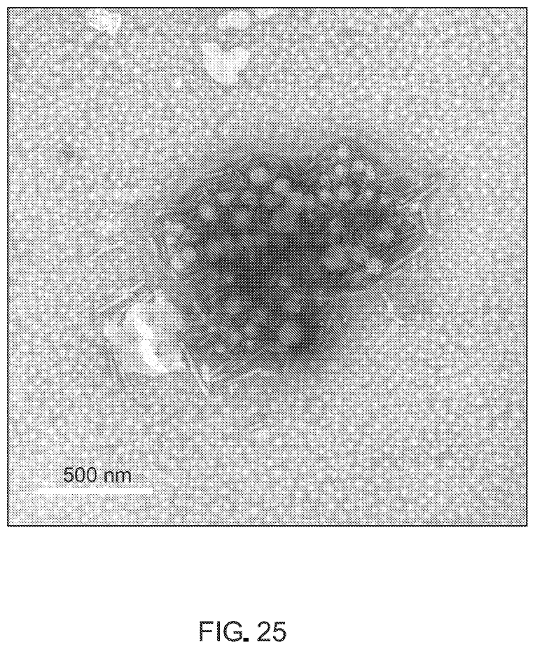

Experiments were conducted to investigate the assembly/disassembly phase diagram of TMGMV and determine conditions amenable to AI entrapment. The primary goal was to achieve “breathing” without full disassembly. First, experiments were focused on pH-induced structural changes and AI infusion. In this approach, the process required extensive optimization, and the parameters pH (7 to 8), incubation time (2 to 24 h), protein concentration (100 to 500 AI equivalences per CP), as well as the AI addition intervals (one feed vs. daily increments) were carefully optimized. The latter was a critical parameter: bulk additions led to severe aggregation and insolubility—likely as a result from the hydrophobic AI binding to the nanoparticle surface promoting interparticle association and aggregation (FIG. 25). It was determined that the best results were obtained when the AI was added in daily increments over 10 days; this assured particle stability and AI loading (see below). In brief, 10 equivalences of AI per CP were fed daily at pH 7.5, left stirring overnight, this process repeating for 10 days. After that, the sample was spin filtered to get rid of any excess AL.

DMSO was used to disrupt inter-coat protein interactions as a second approach. For some AI, in particular those that are highly hydrophobic (e.g., ivermectin and fluopyram), this was favorable. The benefits of this approach can be two-fold: increased solubility of AI can lead to a higher effective concentration to drive infusion, and the cosolvent can prevent AI precipitation which interferes with the infusion process. To further improve on this process, the TMGMV preparations were subjected to magnetic stirring and fed from the top of the tube, preventing any short-term spikes in AI concentration that may promote precipitation. For cases which previously showed immediate precipitation under the pH approach, the DMSO approach showed no visible aggregates and therefore was more likely to succeed with infusion. Additionally, the increased agitation, bond disrupting effect of DMSO, and higher concentrations of AI in solution all suggested that infusion should occur more quickly under these conditions if the TMGMV nanoparticles maintained their structure.

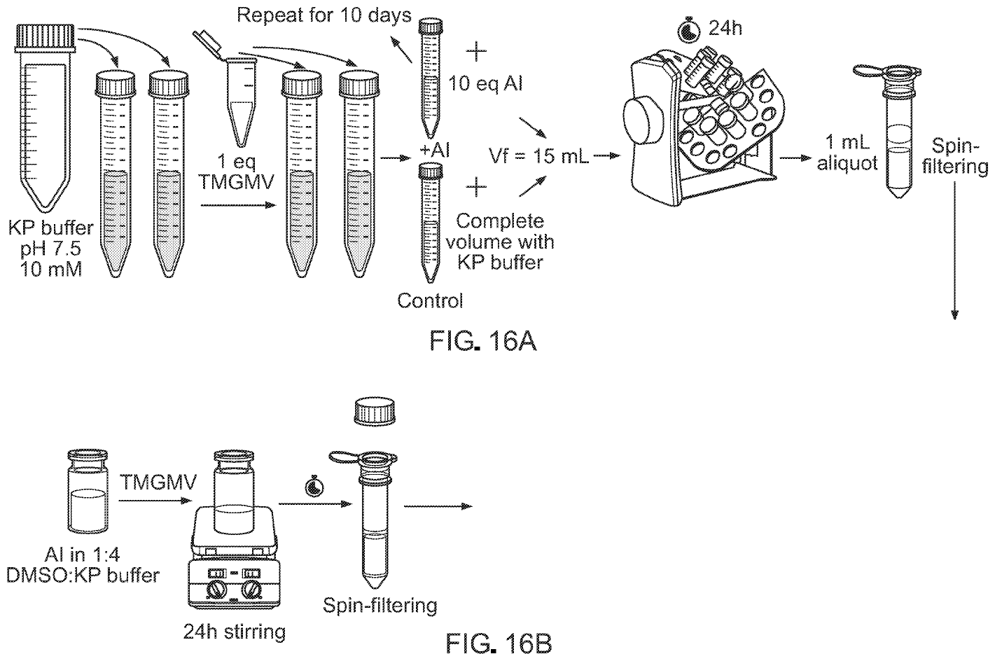

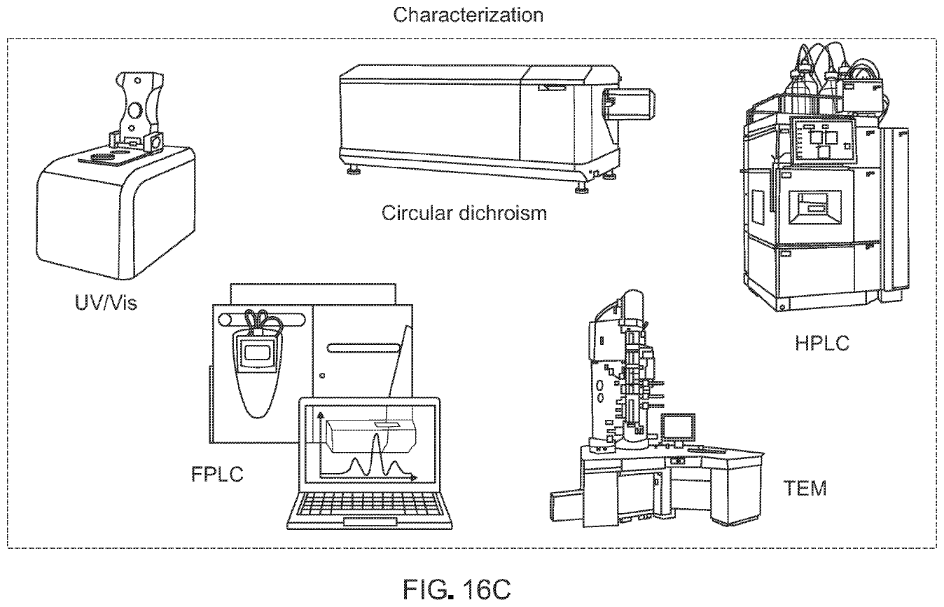

The optimized procedures for each the pH method and the DMSO method are illustrated in FIGS. 16A-16C and described above under the “Methods” section of the Examples.

### Example 6: TEM Characterization of AI-Loaded TMGMV Nanoparticles Prepared Via pH and DMSO Methods

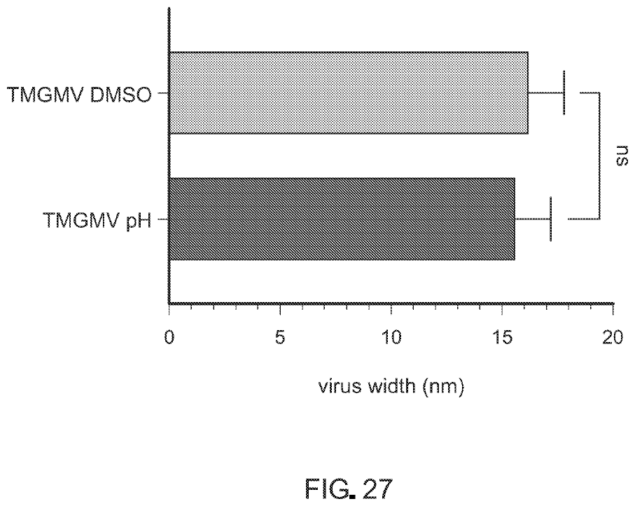

TEM imaging and quantitative TEM imaging analysis of the AI-loaded TMGMV nanoparticles prepared by the pH method and the DMSO method was performed. As evidenced by the TEM images of FIG. 17A, rod-shaped virions were observed in AI-loaded TMGMV nanoparticles prepared by both pH and DMSO methods. Most intriguing was the notable structural change upon AI-loading: AI-laden TMGMV appear swollen and the structural transitions suggest AI entrapment. To gain insights into the degree of structural changes, quantitative TEM image analysis was performed comparing native vs. the AI-laden TMGMV. Based on the negative stain, native virions were 15.7 nm in width in average (±1.9 nm), which is an underestimation to native TMGMV of 18 nm. This may be due to the uranyl acetate negative staining yielding a heavy shadow on the borders of the virion, apparently reducing its width. While there was no statistical significance between the controls that underwent both methodologies (FIG. 27), for each compound the increment (in comparison to native TMGMV) was different, as well as between methodologies (FIG. 17A). Fluopyram- and ivermectin-loaded TMGMV displayed a maximum width of 18 or 23 nm, respectively (FIG. 17B). This resulted in a 14% increase for fluopyram and a 46% width increase for ivermectin compared to negatively-stained native TMGMV. Meanwhile, clothianidin and rifampicin loading produced 22-27 nm rods, significantly thicker than native TMGMV (FIG. 17B). This resulted in a 65% increase for clothianidin and 73% for rifampicin.

### Example 7: Characterization of the Structure of AI-Loaded TMGMV Nanoparticles Prepared Via pH and DMSO Methods

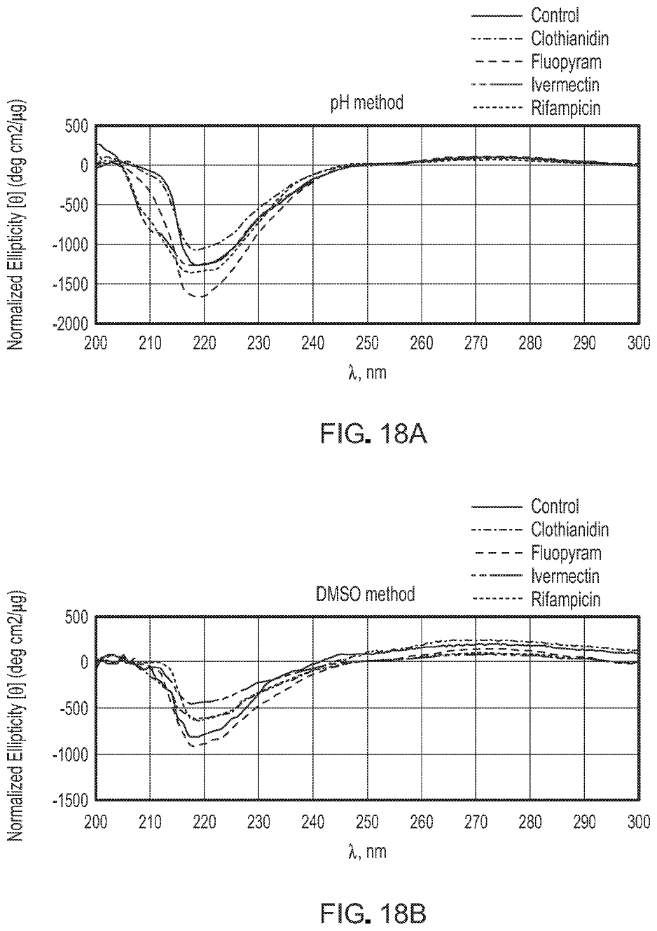

Circular dichroism (CD) was performed to observe any possible alterations in the secondary structure of TMGMV after exposing the particles to breathing and infusion (FIGS. 18A-18B). The effects of structural motifs on circular dichroism are additive and can be challenging to deconvolute. Rather, the differences between spectra of treatment groups can signify whether or not structural changes occurred. The most intense signal for protein or virus CD is around 205-220 nm, which represents the sum of contributions from alpha helices, beta sheets, and aggregation. The shift of the global minimum from 208 nm to 220 nm suggests a larger contribution from aggregation behavior or alpha helical content than beta sheets in both pH and DMSO samples. Other than that, CD indicated no changes in structure—as expected. Coat proteins (CPs) were dissociated to load AI at the interface—the structure was not changed. In the range of 208-220 nm, the AIs tested all showed similar molar ellipticity profiles, suggesting there were no differences in the secondary structure. In the near UV range, a similar trend holds with the AIs, where each shares a general shape of signal profile. These results suggest that both the pH and DMSO approaches for breathing did not significantly alter the secondary structure of TMGMV. In summary, the modest pH elevation and relatively low volume fraction of DMSO used for these breathing experiments were not expected to alter the secondary structure of the virus particles, but rather the tertiary structure to allow inter-coat protein loading.

Size exclusion chromatography (SEC) was performed to further verify structural integrity of the AI-laden TMGMV particles during post-processing and purification. SEC measurements showed no significant difference between native and AI-laden TMGMV for any AI showing the typical elution profile from the Superose6 Increase column with elution at ˜9 mL and an A260:280 ratio of 1.2, indicative of intact TMGMV, where 260 nm indicates RNA absorption and 280 nm protein absorption. In addition to the target pesticide Ais, DOX and Cy5 were used because of their fluorescence properties, DOX and Cy5 exhibit absorbance maxima at 480 nm and 647 nm, respectively. SEC analysis confirmed co-localization of the AI (480 nm and 647 nm) with TMGMV (260/280 nm). The AIs were detected and co-localized at ˜9 mL, by using the absorbance measurements and with the Beer-Lambert law for TMGMV and the AIs (and their molar extinction coefficients), the loading of ˜615 molecules of DOX per TMGMV and ˜80 for Cy5 per TMGMV was determined. Aggregation was observed, especially for Cy5, as well as significant particle dissociation (peaks around ˜20-25 mL) for Dox (FIGS. 26A-26D).

Furthermore, through TEM, rods observed were heterogeneous in length; several disks were apparent indicating partial disassembly and breakage. Also, blob-like structures were observed in the ivermectin sample. These are believed to be ivermectin aggregates of precipitated of the highly hydrophobic ivermectin (FIGS. 17A-17B).

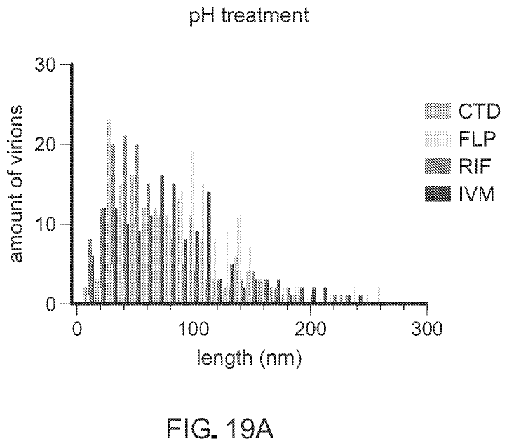

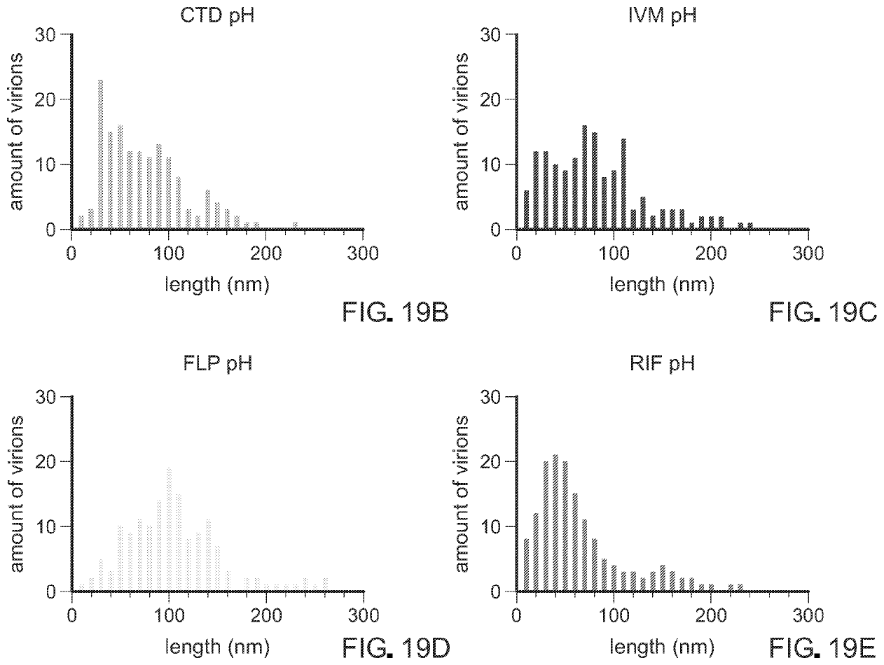

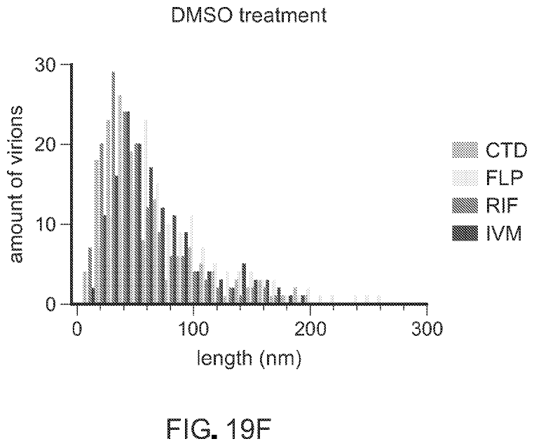

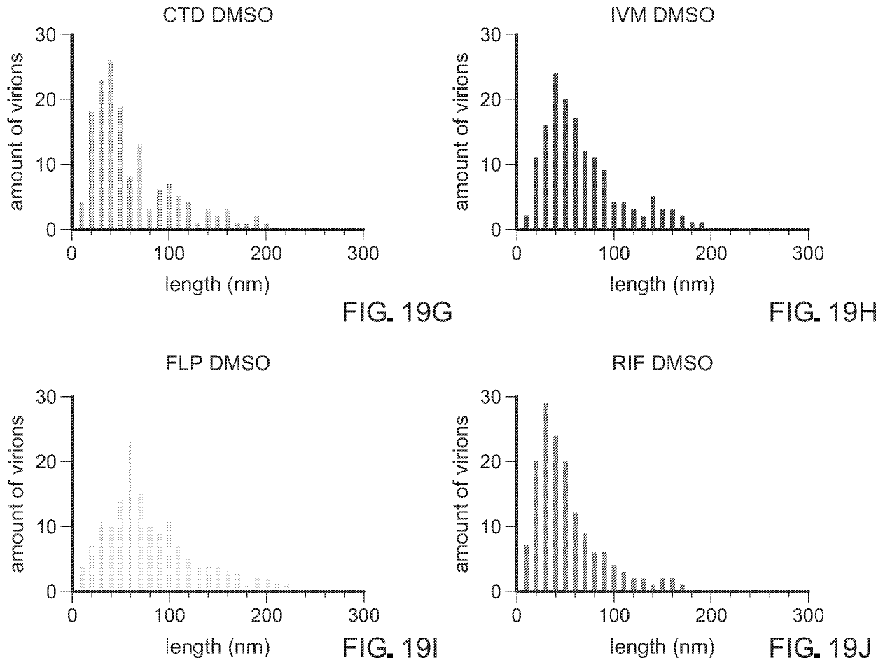

The length of the TMGMV nanoparticles, which were prepared via the pH and DMSO methods, was measured through image analysis. The pH treatment showed slightly less breakage, having a higher distribution of lengths, compared to the DMSO treatment (FIGS. 19A-19J). DMSO treatment showed the majority of the particles below 100 nm. However, there was not a significant difference within AIs, neither between treatments nor between compounds.

### Example 8: Quantification of the Loaded AI in TMGMV Nanoparticles Prepared Via pH and DMSO Methods

The loaded AI was quantified using HPLC (Table 1). Using the pH-based method for infusion, clothianidin and rifampicin showed successful loading after 10 days of batch loading the AI in solution, achieving 1107.55 molecules per virion for clothianidin and 737.66 molecules per virion for rifampicin. In the cases of fluopyram and ivermectin, a large amount of precipitation was observed, which likely stripped the virus from solution and made the AI inaccessible for diffusion into the virus. Fluopyram was calculated to have about 15.82 molecules per virion and ivermectin had 2.89 molecules per virion. When comparing these results to the TEM micrographs and changes in the aspect ratio of the virus after treatment, the amount of loaded AI and changes in morphology appear to be correlated, with clothianidin and rifampicin having the most enlarged virus particles and more appreciable changes in particle width than fluopyram. Ivermectin loading does appear to have significant changes in morphology and was calculated to have wider particles after treatment, although the amount of loaded AI was lower than fluopyram. This may be attributed to challenges in extraction of ivermectin or molecular properties of ivermectin that permanently distort the structure of TMGMV without permanent loading of the AI.

Using the DMSO method for infusion over a 24 hour period, the loading behavior of the AI molecules was improved in most cases. Fluopyram had an 11.7-fold increase in loading using DMSO, achieving 185.59 molecules per virion. A similar result was observed for ivermectin, with a 21.3-fold increase to 61.63 molecules per virion. Clothianidin had about 10% less loading in the 24 hour period using DMSO, reaching 995 molecules per virion. It is possible that a higher clothianidin concentration in solution or a longer infusion period would continue to improve this number and approach the 10-day value for pH-based infusion. Rifampicin loading was improved 1.5-fold using the DMSO method, reaching 1104 molecules per virion.

TABLE 1
Quantification of AI in samples by HPLC.

| | Fluopyram | | Clothianidin | | Ivermectin | | Rifampicin | |
|---|---|---|---|---|---|---|---|---|
| | pH | DMSO | pH | DMSO | ph | DMSO | ph | DMSO |
| Detected in sample | 0.05 μg/mL | 0.61 μg/mL | 2.29 μg/mL | 2.06 μg/mL | 0.02 μg/mL | 0.44 μg/mL | 4.9 μg/mL | 7.52 μg/mL |
| Molecules per virion | 15.82 | 185.59 | 1107.55 | 995 | 2.89 | 61.63 | 737.66 | 1104.2 |
| Molecules per CP | 0.007 | 0.087 | 0.52 | 0.47 | 0.001 | 0.029 | 0.35 | 0.51 |

These data show that DMSO improves the timeline of infusion dramatically compared to the pH-based approach. Morphologically, AI-infused TMGMV nanoparticles look nearly identical using the 10-day pH approach versus the one-day DMSO approach, showing particle integrity is not at risk using 20% DMSO. Reducing the time to achieve infusion to one day also greatly improved the synthesis yield, as fewer particles degraded or precipitated out of solution. Because the DMSO cosolvent also mitigates AI precipitation, the effective concentration of AI for infusion remains higher and drives the molecules into TMGMV. The DMSO concentration, infusion time, mixing rates, and solution concentration of AI versus virus may be further optimized.

When infusing molecules into the rod-shaped TMGMV, several factors led the DMSO approach to be more productive. As previously mentioned, DMSO keeps the solubility of the AI in aqueous buffer higher, thus preventing the precipitation of the AI and potential coprecipitation of the virus. This benefit is two-fold, as precipitated AI cannot diffuse into virus particles and precipitated virus particles cannot be recovered from this process. Additionally, the dropwise addition of AI under magnetic stirring prevents pockets of insoluble concentrations of AI that drive precipitation, as the solution remains well-mixed throughout the entire process. In using 20% v/v DMSO, it seems a balance of structural distortion of TMGMV has been achieved that allows penetration of the AI between the coat proteins of TMGMV. In some embodiments, inter-coat protein loading of AIs in rod-shaped viruses conducted using DMSO results in a 10-fold reduction of synthesis time. This is compounded by the improved synthesis yield by not losing particles in the precipitate and by enabling loading of fluopyram and ivermectin into TMGMV.

### Example 9: Characterization of Molecular Properties of Active Ingredients and TMGMV Nanoparticles

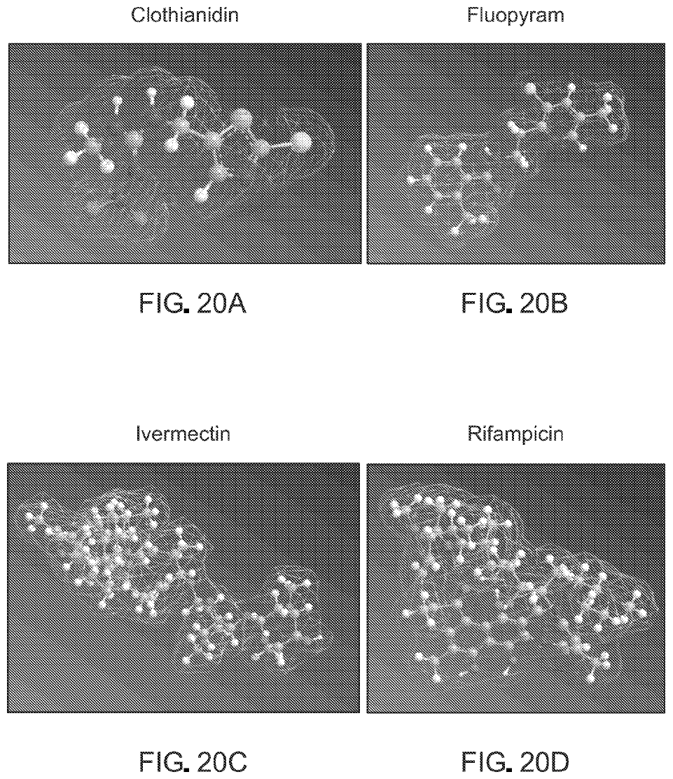

To gain a better understanding of the molecular properties that lead to inter-coat protein loading of AIs, their aqueous and organic partition coefficients (log P), molecular weights, and surface charge distributions were compared. A summary of these properties can be found in Table 2 and FIGS. 20A-20D. Of the AIs that were loaded in TMGMV nanoparticles, ivermectin and fluopyram had the highest log P values of 4.4 and 3.33, respectively. Clothianidin and rifampicin had values of 1.3 and 2.4, respectively. The values for ivermectin and fluopyram indicate the molecules are highly water insoluble, which matches well to what was observed in the loading experiments. This could explain why the same effective morphology changes using these AIs was achieved within 1 day using DMSO versus 10 days for the pH approach, as the effective concentration of AI in solution was much higher. Another factor to consider regarding infusion efficiency is their size. It is possible that larger molecules could have steric hindrance when entering the spaces between coat proteins during these measurements. When the changes in particle width using both approaches were analyzed compared to their molecular weights, it was observed that clothianidin (249.68 Da) had the largest change in width and fluopyram (396.71 Da) had the smallest change in width. Rifampicin (822.94 Da) and ivermectin (875.1 Da) had intermediate values for changes in width. There is no clear trend in this set based on molecular weight, so AI size does not seem to be the limiting factor for loading using this approach.

TABLE 2
Comparison of the AIs aqueous and organic partition
coefficients (logP) and molecular weights.

| Molecule | MW (Da) | logP |
|---|---|---|
| Cyanine 5 | 653.78 | 3.84 |
| Doxorubicin | 543.52 | 0.02 |
| Fluopyram | 396.71 | 3.33 |
| Clothianidin | 249.68 | 1.3 |
| Ivermectin | 875.1 | 4.4 |
| Rifampicin | 822.94 | 2.4 |

Beyond the range of small molecules, steric hindrance would be expected to dominate. From the electron density plots of the AIs, it was observed that ivermectin and rifampicin have large regions with no charge and small regions of small charge that are largely separated, creating mildly amphiphilic molecules. On the contrary, fluopyram and clothianidin are much smaller and have a higher surface area of charge. Because TMGMV is zwitterionic in nature but also contains many hydrophobic interfaces, it is challenging to isolate the predicted changes in morphology to a single physicochemical interaction. The amphiphilic, charged, compact, and flexible structure of clothianidin may all work together to alter the morphology of TMGMV.

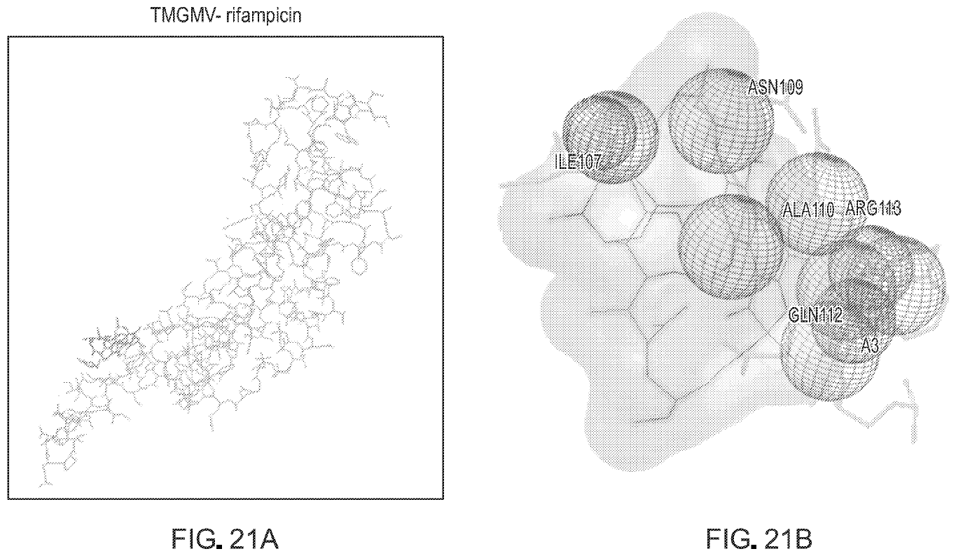

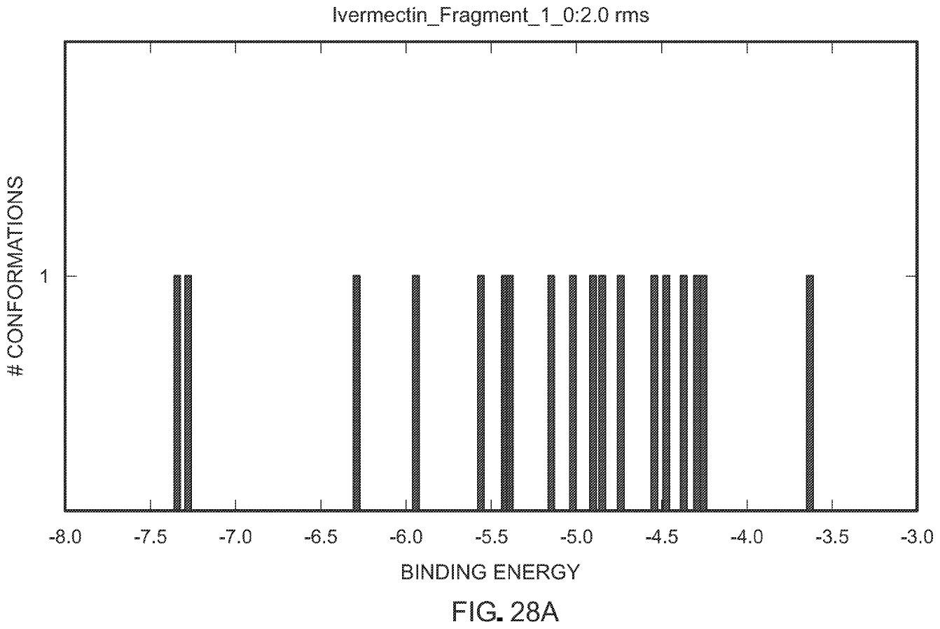

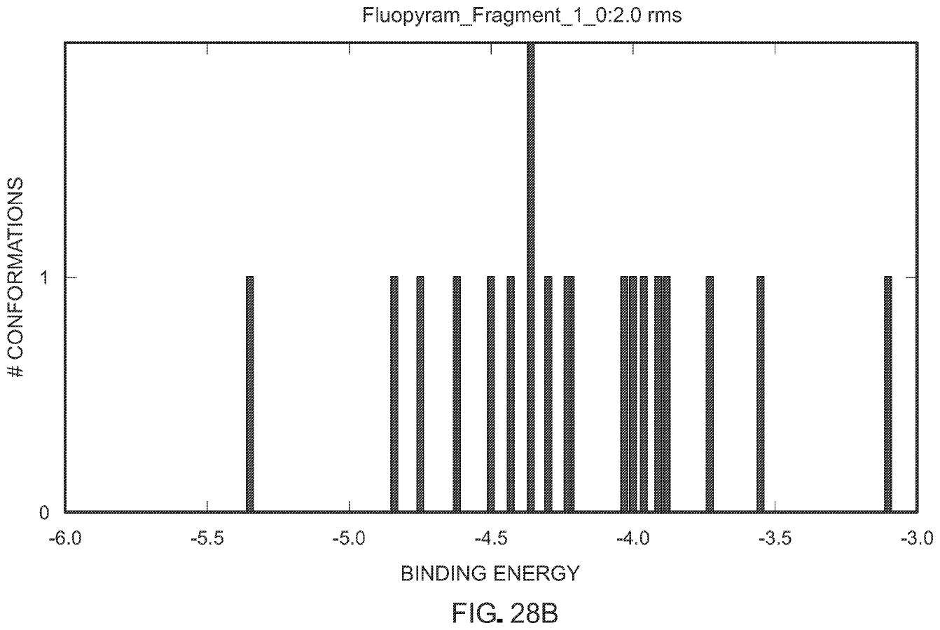

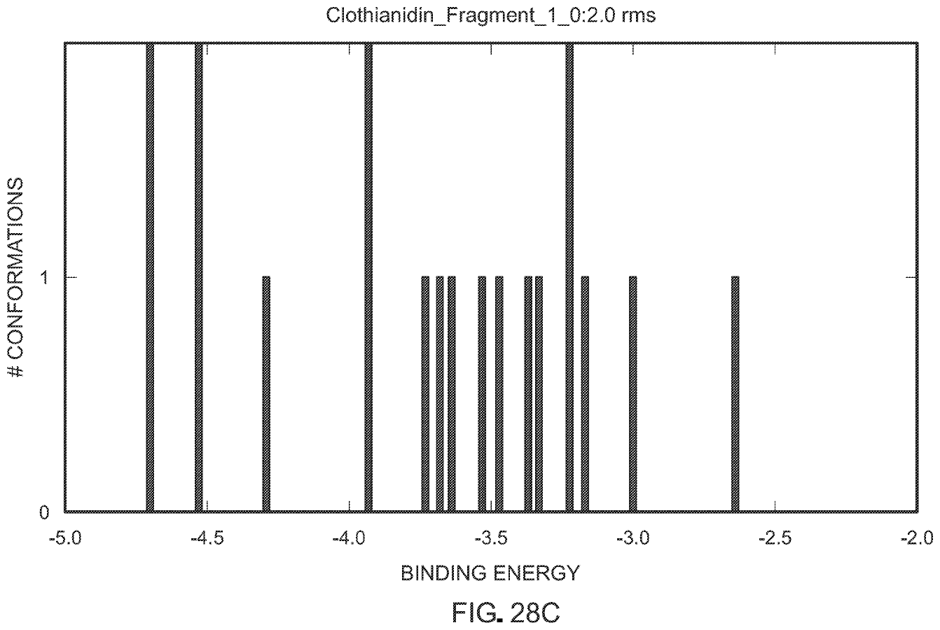

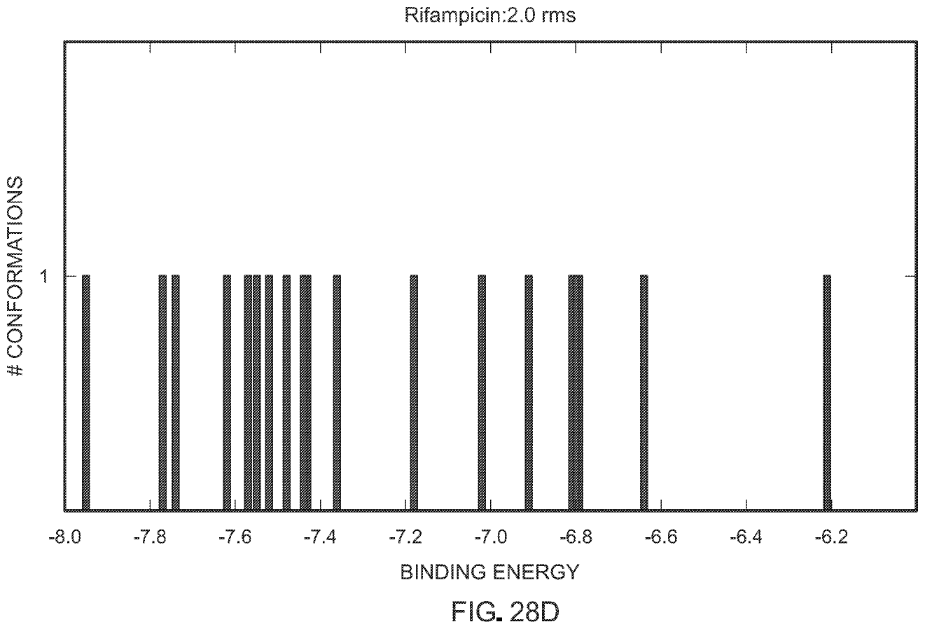

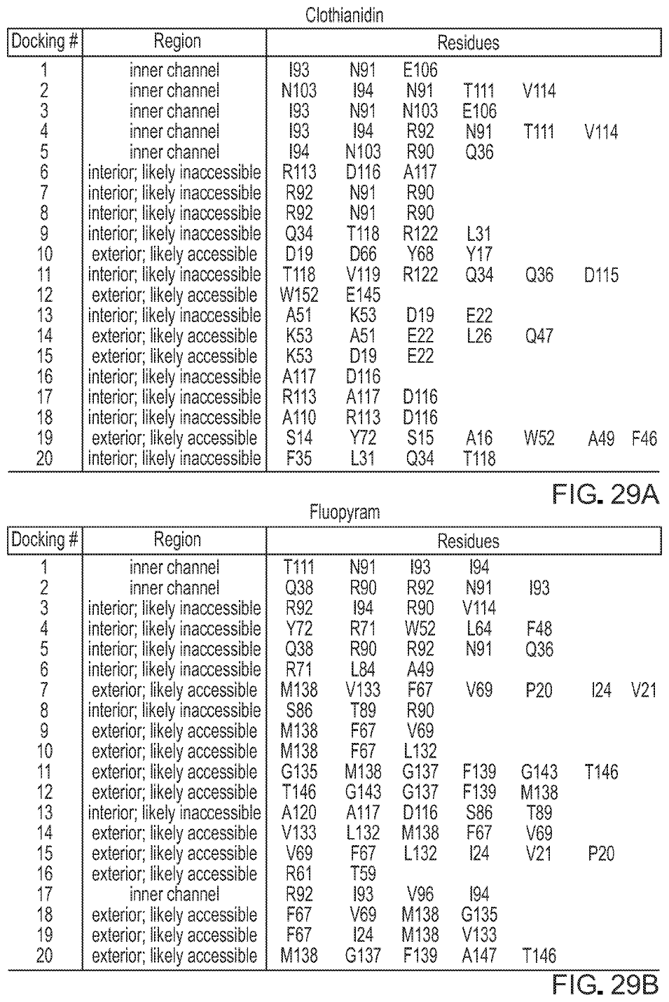

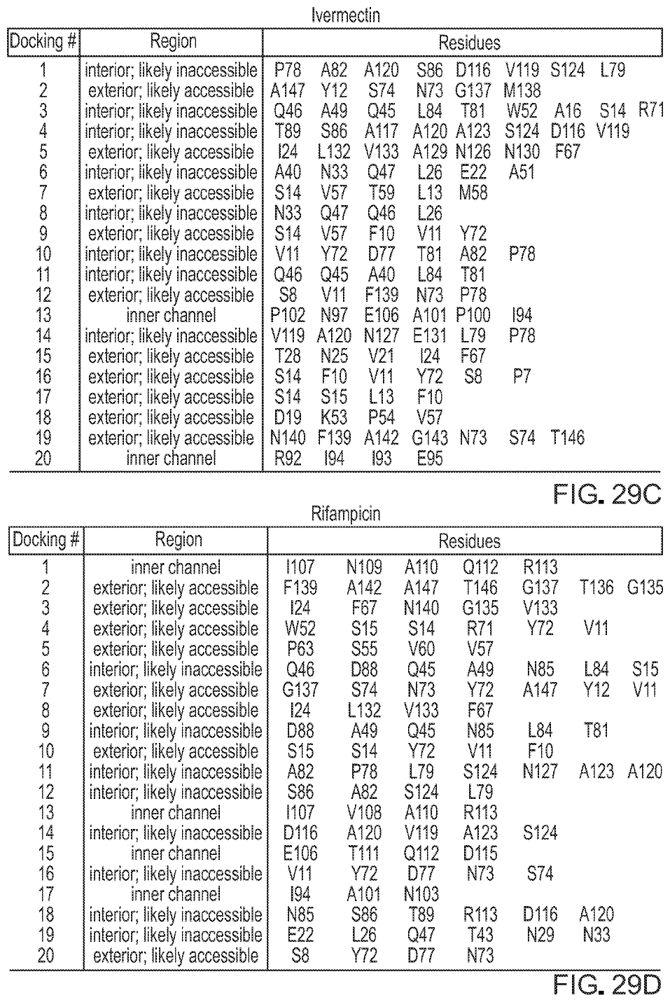

To gain some insight into how the AIs interact with the coat protein surface, molecular docking experiments of TMGMV coat proteins (CP) (PDB: 1VTM) and the four AIs were conducted. In these analyses, the top 20 docking conformations were analyzed for their binding energy and the residues involved in stabilizing the AI. These data do not suggest the AIs are proper ligands for TMGMV CP, but rather identify putative residues that may be implicated in inter-coat protein loading. True ligand interactions have been reported for heats of binding greater than 8 kcal mol⁻¹, while a majority of these interactions fall within 3-8 kcal mol⁻¹. FIGS. 29A-29D summarize the regions of binding, their function for TMGMV, and the residues specifically identified to stabilize the AIs. FIGS. 21A-21B, 22A-22B, 23A-23B, and 24A-24B show examples of docked AIs on TMGMV and the implicated residues, and FIGS. 28A-28D show the heats of binding for each conformation as calculated by Autodock 4. From the simulated docking, it was observed that of the 20 best binding sites on TMGMV CP, all 4 AIs have many sites that are likely inaccessible. Depending on the mechanism of separation of TMGMV CPs (between CPs versus between disks), there are up to 10 accessible sites for ivermectin, 8 for rifampicin, 11 for fluopyram, and 5 for clothianidin. The binding energy distribution shows rifampicin has the highest heats of binding to the surface, followed by ivermectin, then fluopyram and clothianidin. Despite a high number of potential binding sites, ivermectin is a very large molecule and would require a high degree of separation of CPs to intercalate into the virion. Its relatively high affinity may manifest in transient surface binding which can disrupt inter-CP bonds, explaining the widening of TMGMV in the presence of ivermectin. Ultimately, the ivermectin is not detectable during quantification, suggesting it does not stay bound to TMGMV. Rifampicin has the highest heat of binding to TMGMV CP, loads well onto TMGMV, and induces morphological changes on TMGMV. It shows improved loading in the presence of DMSO compared to the pH approach, suggesting the structural changes induced by DMSO allow this relatively large molecule access to binding sites. Despite having 11 potential binding sites, fluopyram also had some of the lowest heats of binding and had the highest affinities for the inner channel. Because this molecule is insoluble and relatively small, it may preferentially partition to the inner channel than to load between the CPs. Clothianidin had 5 accessible sites on the exterior according to the docking model, but also had some of the lowest binding energies. However, its relatively small size and surface charge distribution may have aided in its binding and disruption of structure between TMGMV CPs. Clothianidin demonstrated some of the highest loading by HPLC and largest differences in virion width, suggesting the properties of this molecule make it well suited for this approach. With more robust docking analysis and a larger library of small molecules to load between TMGMV CPs, it may be possible to pinpoint molecular properties of the AIs and individual residues of TMGMV CP that are implicated in these binding events.

### Example 10: TMGMV Nanoparticles Prepared Via pH and DMSO Methods Entrap and Non-Covalently Load Target Molecules

The viral nanoparticle (VNP) AI loading methods described herein not only showed a novel and interesting morphological change in the virus, but also highlighted how these methods can be used for entrapping and non-covalently loading target molecules to the virus and be used as delivery systems. Careful adjustment of solution conditions such as pH and DMSO concentration allow TMGMV to “breathe,” thereby creating structural changes and altering the interactions between structural motifs. These changes enabled AI loading and entrapment in the newly formed pockets, greatly improving the electrostatic loading capacity of TMGMV. The structural distortions in the presence of AI resulted in widening of the minor axis of TMGMV, which correlated to the degree of AI loading. These changes in particle size may simplify on-line measurements during VNP preparation to track the degree of loading in real time.

Both pH and DMSO methodologies showed equal entrapment of rifampicin and clothianidin molecules during viral “breathing,” with up to 1000 AI per TMGMV loaded. However, the DMSO methodology helped in the loading of ivermectin and fluopyram, which formed insoluble precipitates in the absence of DMSO and did not show successful AI entrapment using the pH strategy. Importantly, the DMSO strategy loaded AIs up to 10-fold faster than the pH strategy under the tested conditions, though there are no significant differences in particle integrity between the two conditions. Additional refinement of the “breathing” conditions can help to precisely pinpoint phase transitions of TMGMV, which could result in achieving a higher loading, or the loading of bigger or several different molecules. Overall, the experiments conducted further enlightened TMGMV as a versatile nanotechnological platform for cargo delivery.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-71. (canceled)

72. A nanoparticle comprising:
a tobamovirus; and
one or more active ingredients (AIs) that are non-covalently conjugated to the tobamovirus,
wherein the tobamovirus comprises one or more coat proteins that reversibly and partially dissociate in response to an external factor.

73. The nanoparticle of claim 72, wherein the one or more coat proteins reversibly and partially dissociate to form one or more pores, wherein the one or more AIs are non-covalently conjugated to and entrapped within the one or more pores of the tobamovirus, or wherein the one or more AIs are intercalated in the one or more coat proteins of the tobamovirus.

74. The nanoparticle of claim 73, wherein the one or more AIs are not chemically altered, wherein the external factor is a change in pH, wherein the external factor is the presence of a solvent, or wherein the solvent is a polar, aprotic solvent.

75. The nanoparticle of claim 72, wherein the tobamovirus is rod-shaped, wherein the nanoparticle has a width that is larger than the width of a reference tobamovirus, or wherein the reference tobamovirus has a width of 15, 16, 17, or 18 nm.

76. The nanoparticle of claim 75, wherein the width of the nanoparticle is 2%-105% larger than that of the width of the reference tobamovirus.

77. The nanoparticle of claim 72, wherein the one or more AIs comprises one or more of a drug, pesticide, or a small molecule.

78. The nanoparticle of claim 77, wherein the pesticide comprises a water-insoluble organic compound, a hydrophilic organic compound, an insecticide, a herbicide, a fungicide, an acaricide, an algicide, an antimicrobial agent, biopesticide, a biocide, a disinfectant, a fumigant, an insect growth regulator, a plant growth regulator, a miticide, a microbial pesticide, a molluscide, a nematicide, an ovicide, a pheromone, a repellent, a rodenticide, a defoliant, a desiccant, a safener, or any combination thereof.

79. The nanoparticle of claim 77, wherein the pesticide comprises a benzoyl urea, such as novaluron, lufenuron, chlorfluazuron, flufenoxuron, hexaflumuron, noviflumuron, teflubenzuron, triflumuron and diflubenzuron; a carbamate; a pyrethroid, such as cyhalothrin and isomers and isomer mixtures thereof, lambda-cyhalothrin, deltamethrin, tau-fluvalinate, cyfluthrin, beta-cyfluthrin, tefluthrin, and bifenthrin; an organophosphate, such as azinfos-methyl, chlorpyrifos, diazinon, endosulfan, methidathion; a neonicotinoid; a phenylpyrazole, such as imidacloprid, acetamiprid, thiacloprid, dinotefuran, thiamethoxam, and fipronil; a conazole, such as epoxiconazole, hexaconazole, propiconazole, prochloraz, imazalil, triadimenol, difenoconazole, myclobutanil, prothioconazole, triticonazole, and tebuconazole; a morpholine, such as dimethomorph, fenpropidine, and fenpropimorph; a strobilurin, such as azoxystrobin, kresoxim-methyl, and analogues thereof; a phthalonitrile, such as chlorothalonil; a mancozeb; a fluazinam; a pyrimidine, such as bupirimate; an aryloxyphenoxy derivative; an aryl urea; an aryl carboxylic acid; an aryloxy alkanoic acid derivative, such as clodinafop-propargyl and analogues thereof, fenoxaprop-p-ethyl and analogues thereof, propaquizafop, quizalafop and analogues thereof; a dintroaniline, such as pendimethalin and trifluralin; a diphenyl ether, such as oxyfluorfen; an imidazolinone; a sulfonylurea, such as chlorsulfuron, nicosulfuron, rimsulfuron, tribenuron-methyl; a sulfonamide; a triazine; and a triazinone, such as metamitron.

80. The nanoparticle of claim 77, wherein the drug is a chemotherapeutic drug, an antiparasitic drug, an antibiotic drug, or an immunomodulator; or wherein the drug is a hydrophilic drug or a hydrophobic drug.

81. The nanoparticle of claim 72, wherein the nanoparticle comprises about 1 to about 1500 AI molecules per tobamovirus.

82. The nanoparticle of claim 72, wherein the tobamovirus is a Tobacco Mild Green Mosaic Virus (TMGMV), or wherein the tobamovirus is a Tobacco Mosaic Virus (TMV).

83. A composition comprising the nanoparticle of claim 72, wherein the composition demonstrates a soil distribution and/or soil mobility of at least 5, 10, 15, 20, 21, 22, 23, 24, 25, 26, 27, 28, 29, 30, 21, 32, 33, 34, 35, 36, 37, 38, 39, or 40 cm from point of application, wherein the composition further comprises an excipient, or wherein the excipient is a buffer or water.

84. A pharmaceutical composition comprising the nanoparticle of claim 72.

85. A method of making a nanoparticle comprising a tobamovirus and one or more active ingredients (AIs), the method comprising:
providing isolated a tobamovirus to a buffer having a pH of about 5 to 9 to create a tobamovirus-buffer;
adding one or more AIs to the tobamovirus-buffer more than once, thereby creating the nanoparticle; and
purifying the nanoparticle in a solution having a pH of about 5 to 9,
wherein, the one or more AIs are non-covalently conjugated to the tobamovirus, and
wherein the tobamovirus comprises one or more coat proteins that reversibly and partially dissociate in response to a change in pH.

86. The method of claim 85, wherein the one or more AIs are added at least once a day for at least 1, 2, 3, 4, 5, 6, 7, 8, 9, 10, 11, 12, 13, 14, 15, 16, 17, 18, 19, 20, or 21 days, wherein the buffer has a pH of about 7 to 7.5, 7.5 to 8, 7 to 8, 8 to 8.5, 8.5 to 9, or 8 to 9, wherein the solution has a pH of about 6.9, 7.0, 7.1, 7.2, or 7.3, or wherein the change in pH is about 0.5 to 1, about 0.5 to 2, 0.5 to 3, 1 to 2, or 1 to 3.

87. The method of claim 85, further comprising adding a solvent at a concentration of about 15% (v/v) to about 25% (v/v).

88. A method of treating cancer in a subject in need thereof, the method comprising:
administering a nanoparticle comprising:
a tobamovirus; and
one or more active ingredients (AIs) that are non-covalently conjugated to the tobamovirus,
wherein the tobamovirus comprises one or more coat proteins that reversibly and partially dissociate in response to an external factor, and
wherein the nanoparticle is administered in an effective amount.

89. The method of claim 88, wherein the cancer wherein the cancer comprises breast cancer, ovarian cancer, glioma, gastrointestinal cancer, prostate cancer, carcinoma, lung carcinoma, hepatocellular carcinoma, testicular cancer, cervical cancer, endometrial cancer, bladder cancer, head and neck cancer, lung cancer, gastro-esophageal cancer, gynecological cancer, or any combination thereof.

90. A method of treating an infection in a subject in need thereof, the method comprising:
administering a nanoparticle comprising:
a tobamovirus; and
one or more active ingredients (AIs) that are non-covalently conjugated to the tobamovirus,
wherein the tobamovirus comprises one or more coat proteins that reversibly and partially dissociate in response to an external factor, and
wherein the nanoparticle is administered in an effective amount.

91. The method of claim 90, wherein the infection is a bacterial infection, a viral infection, a fungal infection, a parasitic infection, or any combination thereof.
